(19) United States
(12) Patent Application Publication
(10) Pub. No.: US 2023/0040370 A1
(43) Pub. Date: Feb. 9, 2023

(54) GRAPHENE BIOSCAFFOLDS AND THEIR USE IN CELLULAR THERAPY

(71) Applicant: The Board of Trustees of the Leland Stanford Junior University, Stanford, CA (US)

(72) Inventors: Avnesh S. Thakor, Palo Alto, CA (US); Mehdi Razavi, Palo Alto, CA (US)

(21) Appl. No.: 17/815,485

(22) Filed: Jul. 27, 2022

Related U.S. Application Data
(60) Provisional application No. 63/228,236, filed on Aug. 2, 2021.

(51) Int. Cl. A61L 27/08; A61L 27/18; A61L 27/38; A61P 3/10; A61L 27/54; A61L 27/56; A61K 31/573; A61K 35/28; A61K 35/39; A61K 9/70; A61L 27/58

(52) U.S. Cl. CPC A61L 27/08; A61L 27/18; A61L 27/3834; A61L 27/3804; A61P 3/10; A61L 27/54; A61L 27/56; A61K 31/573; A61K 35/28; A61K 35/39; A61K 9/7007; A61L 27/58; A61L 2300/43; A61L 2300/606; C23C 16/04

(57) ABSTRACT

A bioscaffold comprising a graphene matrix for use in cellular therapy is disclosed. In particular, a bioscaffold having a coating of dexamethasone on a three-dimensional graphene matrix is provided, wherein the bioscaffold elutes dexamethasone to reduce inflammatory responses following implantation of the bioscaffold in a subject. Having the dexamethasone released locally in the vicinity of the bioscaffold avoids the systemic side effects from conventional intravenous delivery while allowing the dexamethasone to modulate the inflammatory milieu within the transplantation microenvironment.

### CROSS-REFERENCE TO RELATED APPLICATION

This application claims benefit under 35 U.S.C. § 119(e) of provisional application 63/228,236, filed Aug. 2, 2021, which application is hereby incorporated by reference in its entirety.

### STATEMENT REGARDING FEDERALLY SPONSORED RESEARCH OR DEVELOPMENT

Type 1 This invention was made with Government support under contracts DK116074, DK119293, and NS069375 awarded by the National Institutes of Health. The Government has certain rights in the invention.

### BACKGROUND OF THE INVENTION

Type 1 diabetes mellitus (T1 DM) affects 1.4 million people in the United States and 30 million globally. The commercial availability of insulin in the 1920s transformed T1 DM from an untreatable condition to one that can be treated with daily injections of insulin. Conventional insulin therapy, however, can only keep blood glucose levels within a broad range and cannot respond dynamically to second-by-second changes in blood glucose variability. Tight glycemic control has been shown to significantly reduce the incidence of secondary complications of T1 DM, including renal failure, blindness and heart disease. Even with intensive insulin therapy, however, T1 DM is associated with a 200-300% increase in life threatening hypoglycemia. Hence, if a treatment could be developed to restore glucose homeostasis in an automated and self-regulating manner, this would revolutionize the quality of life for patients with T1 DM.

One promising approach to treating T1 DM is pancreatic islet transplantation, where islets from the pancreas of a donor are isolated and then placed into a recipient patient with T1 DM. Within the first week following islet transplantation, however, 50-70% of transplanted islets are lost, primarily due to hypoxia and the instant blood mediated inflammatory reaction (IBMIR). Therefore, it would be highly beneficial to have improved devices and methods for transplanting cells.

### SUMMARY OF THE INVENTION

A bioscaffold comprising a graphene matrix for use in cellular therapy is disclosed. In particular, a bioscaffold having a coating of dexamethasone on a three-dimensional graphene matrix is provided, wherein the bioscaffold elutes dexamethasone to reduce inflammatory responses following implantation of the bioscaffold in a subject. Having the dexamethasone released locally in the vicinity of the bioscaffold avoids the systemic side effects from conventional intravenous delivery while allowing the dexamethasone to modulate the inflammatory milieu within the transplantation microenvironment.

An implantable graphene bioscaffold that elutes dexamethasone was tested with islets and adipose tissue-derived mesenchymal stem cells (AD-MSCs). In vitro studies demonstrated that the islets in graphene bioscaffolds with dexamethasone had a significantly higher viability and function compared to islets in graphene bioscaffolds without dexamethasone or islets cultured alone. In vivo studies, in which graphene bioscaffolds were transplanted into the epididymal fat pad (EFP) of diabetic mice, demonstrated that when islets and AD-MSCs were loaded into graphene bioscaffolds with dexamethasone, glycemic control was restored immediately after transplantation, with these islets also showing a faster response to glucose challenges (see Examples). Hence, this combination approach of using a graphene bioscaffold that can be functionalized for local delivery of dexamethasone into the surrounding microenvironment, together with AD-MSC therapy, can significantly improve the survival and function of transplanted islets.

In one aspect, a bioscaffold is provided, the bioscaffold comprising: a) a three-dimensional graphene matrix, wherein the graphene matrix comprises a plurality of macropores and micropores; and b) a coating comprising dexamethasone.

In certain embodiments, the bioscaffold further comprises a polydopamine nanolayer on the surface of the graphene matrix, wherein the polydopamine nanolayer is functionalized with the dexamethasone.

In certain embodiments, the graphene matrix has a porosity ranging from 55% to 95%, including any porosity within this range such as 55%, 57%, 59%, 61%, 63%, 65%, 67%, 69%, 71%, 73%, 75%, 77%, 79%, 81%, 83%, 85%, 87%, 89%, 91%, 93%, or 95%. In some embodiments, the graphene matrix has a porosity of about 80 percent.

In certain embodiments, the macropores have an average diameter ranging from about 400 μm to about 800 μm, as measured by scanning electron microscopy, including any average diameter within this range such as 400 μm, 420 μm, 440 μm, 460 μm, 480 μm, 500 μm, 520 μm, 540 μm, 560 μm, 580 μm, 600 μm, 620 μm, 640 μm, 660 μm, 680 μm, 700 μm, 720 μm, 740 μm, 760 μm, 780 μm, or 800 μm.

In certain embodiments, the micropores have an average diameter of 200 μm or less as measured by scanning electron microscopy. In some embodiments, the micropores have an average diameter ranging from about 100 μm to about 200 μm, as measured by scanning electron microscopy, including any average diameter within this range such as 100 μm, 110 μm, 120 μm, 130 μm, 140 μm, 150 μm, 160 μm, 170 μm, 180 μm, 190 μm, or 200 μm.

In certain embodiments, the dexamethasone is at a concentration of about 0.25 to about 1 weight/volume percent (w/v %) in the bioscaffold, including any concentration within this range such as 0.25 w/v %, 0.30 w/v %, 0.35 w/v %, 0.4 w/v %, 0.45 w/v %, 0.50 w/v %, 0.55 w/v %, 0.60 w/v %, 0.65 w/v %, 0.70 v, 0.75 w/v %, 0.80 w/v %, 0.85 w/v %, 0.90 w/v %, 0.95 w/v %, or 1.00 w/v %.

In certain embodiments, the bioscaffold has a thickness of from about 0.1 mm to about 25 mm, including any thickness within this range such as 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 8.0 mm, 9.0 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the thickness ranges from about 0.5 mm to about 5 mm.

In certain embodiments, the bioscaffold further comprises one or more drugs, growth factors, angiogenic agents, cytokines, or extracellular matrix components, or a combination thereof.

In certain embodiments, the bioscaffold further comprises therapeutic cells or extracellular vesicles, wherein the therapeutic cells or extracellular vesicles are contained in the macropores. In some embodiments, the therapeutic cells are stem cells, progenitor cells, or mature cells. For example, the stem cells may be induced-pluripotent stem cells or adult stem cells. In some embodiments, the stem cells are mesenchymal stem cells. In some embodiments, the therapeutic cells secrete a cytokine, a chemokine, an antibody, an enzyme, a growth factor, or a hormone. In some embodiments, the therapeutic cells are endocrine cells, exocrine cells, stem cells, or lymphocytes. In some embodiments, the therapeutic cells are genetically modified cells.

In certain embodiments, the therapeutic cells are insulin-secreting cells. In some embodiments, the insulin-secreting cells are pancreatic beta cells or islets obtained from a donor. In some embodiments, the insulin-secreting cells are derived from stem cells or pancreatic progenitor cells. In some embodiments, the therapeutic cells comprise insulin-secreting cells in combination with stem cells. In some embodiments, the stem cells are mesenchymal stem cells.

In certain embodiments, the extracellular vesicles are exosomes, ectosomes, microvesicles, or microparticles derived from a plasma membrane of a cell. In some embodiments, the extracellular vesicles are derived from mesenchymal stem cells.

In another aspect, a method of treating a subject for type 1 diabetes is provided, the method comprising implanting a bioscaffold described herein in the subject at an implantation site.

In certain embodiments, the bioscaffold releases dexamethasone for at least 2 weeks at the implantation site in vivo.

In certain embodiments, the insulin-secreting cells are autologous, allogeneic, or xenogeneic pancreatic beta cells or islets. In some embodiments, the insulin-secreting cells are derived from stem cells or pancreatic progenitor cells.

In certain embodiments, the implantation site is in a kidney, liver, omentum, peritoneum, abdomen, submuscular tissue, or subcutaneous tissue of the subject.

In another aspect, a tissue graft is provided, the tissue graft comprising: a) a bioscaffold described herein; and b) a plurality of therapeutic cells encapsulated within the macropores of the graphene matrix.

In another aspect, a method of producing a tissue graft is provided, the method comprising: a) depositing a plurality of therapeutic cells on a bioscaffold described herein; and b) culturing the deposited therapeutic cells under conditions wherein an effective amount of the therapeutic cells is encapsulated in the macropores of the graphene matrix.

In certain embodiments, the therapeutic cells are insulin-secreting cells. In some embodiments, the insulin-secreting cells are autologous, allogeneic, or xenogeneic pancreatic beta cells or islets. In some embodiments, the insulin-secreting cells are derived from stem cells or pancreatic progenitor cells. In some embodiments, the insulin-secreting cells are pancreatic beta cells or islets obtained from a donor. In some embodiments, the therapeutic cells further comprise stem cells. In some embodiments, the stem cells are mesenchymal stem cells.

In another aspect, a method for making a graphene bioscaffold is provided, the method comprising: a) fabricating a three-dimensional graphene matrix using template-directed chemical vapor deposition; b) coating the surface of the graphene matrix with polydopamine; and c) functionalizing the polydopamine coating with dexamethasone.

In certain embodiments, the method further comprises depositing therapeutic cells or extracellular vesicles on the bioscaffold.

In certain embodiments, the therapeutic cells are insulin-secreting cells. In some embodiments, the insulin-secreting cells are autologous, allogeneic, or xenogeneic pancreatic beta cells or islets. In some embodiments, the insulin-secreting cells are pancreatic beta cells or islets obtained from a donor. In some embodiments, the insulin-secreting cells are derived from stem cells or pancreatic progenitor cells. In some embodiments, the therapeutic cells further comprise stem cells. In some embodiments, the stem cells are mesenchymal stem cells.

In certain embodiments, the method further comprises adding one or more drugs, growth factors, angiogenic agents, cytokines, or extracellular matrix components, or a combination thereof to the bioscaffold.

In another aspect, a method of regulating blood glucose levels in a subject, the method comprising implanting a bioscaffold described herein in the subject at an implantation site. In certain embodiments, the therapeutic cells are insulin-secreting cells. In some embodiments, the insulin-secreting cells are autologous, allogeneic, or xenogeneic pancreatic beta cells or islets. In some embodiments, the insulin-secreting cells are pancreatic beta cells or islets obtained from a donor. In some embodiments, the insulin-secreting cells are derived from stem cells or pancreatic progenitor cells. In some embodiments, the therapeutic cells further comprise stem cells. In some embodiments, the stem cells are mesenchymal stem cells.

In certain embodiments, the implantation site is in a kidney, liver, omentum, peritoneum, abdomen, submuscular tissue, or subcutaneous tissue of the subject.

In certain embodiments, the subject has hyperglycemia or type 1 diabetes.

In another aspect, a bioscaffold for use in treating type 1 diabetes, wherein the bioscaffold comprises: a) a three-dimensional graphene matrix, wherein the graphene matrix comprises a plurality of macropores and micropores; b) a coating comprising dexamethasone; and c) insulin-secreting cells and stem cells, wherein the insulin-secreting cells and stem cells are contained in the macropores.

In certain embodiments, the insulin-secreting cells are autologous, allogeneic, or xenogeneic pancreatic beta cells or islets. In some embodiments, the insulin-secreting cells are pancreatic beta cells or islets obtained from a donor. In some embodiments, the insulin-secreting cells are derived from stem cells or pancreatic progenitor cells. In some embodiments, the therapeutic cells further comprise stem cells. In some embodiments, the stem cells are mesenchymal stem cells.

### BRIEF DESCRIPTION OF THE DRAWINGS

FIGS. 1A-1Q. Bioscaffold fabrication using a template directed CVD method and characterizations: (FIG. 1A) photograph, and (FIG. 1B-1C) a scanning electron microscopy (SEM) image of a nickel foam in low (FIG. 1B) and high (FIG. 1C) magnification; (FIG. 1D-1F) photos of our CVD chamber before (FIG. 1D), during (FIG. 1E), and after (FIG. 1F) the graphene growth on the nickel foam (the arrows indicate the nickel foam placed inside the chamber). The nickel foam was first annealed at 1000° C. for 5 min under Ar and H₂ atmosphere with the flow rates of 500 and 200 s.c.c.m., respectively to clean its surfaces and eliminate the surface oxide layer. The CH₄ gas with the flow rate of 50 s.c.c.m was then introduced into the reaction chamber. After 5 min of reaction-gas mixture flow, the foam was rapidly cooled down to room temperature under Ar and H₂ atmosphere with the flow rates of 500 and 200 s.c.c.m., respectively; (FIG. 1G) schematic representation showing graphene bioscaffolds before and after the transfer process of graphene bioscaffold from nickel-graphene foam. The nickel-graphene foam was first coated with a thin PMMA layer to support the graphene structure and prevent its structural failure when the nickel foams is etched away in next step. Then, the nickel-graphene-PMMA foam was immersed into a mixture of FeCl₃ and HCl at 80° C. for 72 h to dissolve the nickel. Finally, graphene bioscaffolds were obtained by dissolving the PMMA with acetone; (FIG. 1H-1O) photos (FIGS. 1H, 1L; showing a self-standing 3D porous structure) and SEM images (FIGS. 1I-1K, 1M-1O); showing a monolith of continuous and porous structure, which copied and inherited the interconnected 3D structure of the nickel foam template) of thin (FIGS. 1I-1K) and thick (FIGS. 1M-1O) graphene bioscaffolds. SEM images also indicate the Dex microparticles that has been immobilized on the surface of graphene bioscaffolds (FIGS. 1K, 1O); (FIGS. 1P-1Q) bioscaffold physicochemical characterization include Raman spectroscopy (FIG. 1P) and XPS (FIG. 1Q). Photo Credit: Mehdi Razavi, Stanford University.

FIGS. 2A-2B. Bioscaffold Coating with Dex: (FIG. 2A) SEM images from graphene-Dex bioscaffolds with increasing Dex concentrations from 0-1 wt/v % showing Dex particles that have been attached onto the surface of graphene bioscaffolds and by increasing the concentration of Dex from 0.25 to 1 wt/v %, more Dex particles are seen; (FIG. 2B) The Dex release profile showing the ability of graphene-Dex bioscaffolds to release Dex for at least 14 days, with the release rate significantly increasing as the concentration of Dex increases from 0.25 to 1 wt/v % (p<0.05). Significant differences: ^{a}p<0.05: graphene-0.25 wt/v % Dex bioscaffolds vs. graphene-0.5 wt/v % Dex bioscaffolds or graphene-1 wt/v % Dex bioscaffolds, ^{b}p<0.05: graphene-0.5 wt/v % Dex bioscaffolds vs. graphene-1 wt/v % Dex bioscaffolds; *day 1 vs. days 3, 7, 14.

FIGS. 3A-3G. Bioscaffold interactions with pancreatic islets in vitro: (FIG. 3A) SEM images of the top surface, and center, of our graphene-Dex bioscaffold seeded with islets; (FIG. 3B) Bright-field images of islets cultured in conventional culture plates; (FIG. 3C) Confocal images of islets cultured in culture plates (islets only) or in graphene alone bioscaffolds or graphene-Dex bioscaffolds with 0.25, 0.5, and 1 wt/v % Dex at day 7. Green represents live cells and red represents dead cells; Results of (FIG. 3D) Live/Dead, (FIG. 3E) MTT, and (FIG. 3F-3G) GSIS assays for islets alone and islets in graphene bioscaffolds without, and with, Dex at day 7. Red: dead cells stained with PI. Green: live cells stained with FDA. Significant differences: (FIGS. 3D-3G) ^{a}p<0.05: islets alone vs. graphene and graphene-0.25, 0.5 and 1 wt/v % Dex bioscaffolds; ^{b}p<0.05: graphene bioscaffolds vs. graphene-0.25, 0.5 and 1 wt/v % Dex bioscaffolds; ^{c}p<0.05: graphene-0.25 wt/v % bioscaffolds vs. graphene-0.5 and 1 wt/v % Dex bioscaffolds; ^{d}p<0.05: graphene-0.5 wt/v % bioscaffolds vs. graphene-1 wt/v % Dex bioscaffolds; *low glucose (LG) vs. high glucose (HG).

FIGS. 4A-4G. Bioscaffold interactions with pancreatic islets in vivo: (FIG. 4A) Experimental details of our in vivo experiment and schematic representation of our bioscaffold transplantation in the EFP; Results of (FIG. 4B) non-fasting blood glucose measurements, (FIG. 4C) percentage of normoglycemia, (FIG. 4D) body weight, (FIG. 4E) IPGTT (i.e. changes of fasting blood glucose versus baseline (0 min time point) and (FIG. 4F) results of calculation of area under the curve (AUC_{0-120 min}) of IPGTT curves (i.e. glucose clearance rate); (FIG. 4) Photographs of the transplantation procedure of islets alone and islet:AD-MSCGs units in graphene-0.5 wt/v % Dex bioscaffolds. Photo Credit: Mehdi Razavi, Stanford University. Significant differences: (FIG. 4B-4G) ^{a}P<0.05: islets alone vs. islets in graphene alone bioscaffolds or islets in graphene-0.5 wt/v % Dex bioscaffolds or islet:AD-MSCs units in graphene-0.5 wt/v % Dex bioscaffolds; ^{b}P<0.05: islets in graphene alone bioscaffolds vs. islets in graphene-0.5 wt/v % Dex bioscaffolds or islet:AD-MSCs units in graphene-0.5 wt/v % Dex bioscaffolds; ^{c}P<0.05: islets in graphene-0.5 wt/v % Dex bioscaffolds vs. islet:AD-MSCs units in graphene-0.5 wt/v % Dex bioscaffolds. (FIG. 4B) *P<0.05: baseline (day −2) vs. all other time-points (Two-way ANOVA post-hoc Tukey Test). (FIGS. 4C-4D) *P<0.05: post-transplant week 0 vs. post-transplant week 1, 2, 3, and 4 (Two-way ANOVA post-hoc Tukey Test). (FIG. 4E) *P<0.05: 0 min vs. 30, 60, 90 and 120 min (Two-way ANOVA post-hoc Tukey Test). (FIG. 4F) (One-way ANOVA post-hoc Tukey Test).

FIGS. 5A-5G. Histological and molecular analyses: (FIG. 5A) Representative histological (H&E staining) and immunohistochemical images (insulin, vWF, and TNF-α staining) of the EFP containing islets alone or islets into graphene alone and graphene-0.5 wt/v % Dex bioscaffolds or islet:AD-MSCs units into graphene-0.5 wt/v % Dex bioscaffolds; Red stars=islets; Black arrows=bioscaffolds, Blue arrows=positive (brown) staining; (FIG. 5B) Cytokines expression profile within the EFP tissue; The level of insulin within the (FIG. 5C) EFP and (FIG. 5D) blood serum; Quantification of positive (FIG. 5E) insulin, (FIG. 5F) vWF, and (FIG. 5G) TNF-α staining. Results were analyzed with at least 15-20 islets from 5 different sections through the EFP of each animal. Significant differences: (FIG. 5B) ^{a}P<0.05: islets in graphene alone bioscaffolds vs. islets in graphene-0.5 wt/v % Dex bioscaffolds or islets:AD-MSCs units in graphene-0.5 wt/v % Dex bioscaffolds; ^{b}P<0.05: islets in graphene-0.5 wt/v % Dex bioscaffolds vs. islets:AD-MSCs units in graphene-0.5 wt/v % Dex bioscaffolds; *P<0.05: islets alone (control) vs. islets in graphene alone bioscaffolds or islets in graphene-0.5 wt/v % Dex bioscaffolds or islet:AD-MSCs units in graphene-0.5 wt/v % Dex bioscaffolds (One-way ANOVA post-hoc Tukey Test). (FIG. 5C-5G) ^{a}P<0.05: islets alone vs. islets in graphene alone bioscaffolds or islets in graphene-0.5 wt/v % Dex bioscaffolds or islet:AD-MSCs units in graphene-0.5 wt/v % Dex bioscaffolds; ^{b}P<0.05: islets in graphene alone bioscaffolds vs. islets in graphene-0.5 wt/v % Dex bioscaffolds or islets:AD-MSCs units in graphene-0.5 wt/v % Dex bioscaffolds; ^{c}P<0.05: islets in graphene-0.5 wt/v % Dex bioscaffolds vs. islets:AD-MSCs units in graphene-0.5 wt/v % Dex bioscaffolds.

FIGS. 6A-6F. Bioscaffold biodegradability and biocompatibility: (FIG. 6A) The biodegradation profile and photographic of graphene-0.5 wt/v % Dex bioscaffolds before and after incubation in PBS for 3 months; (FIG. 6B) Photographic, and representative histological (H&E staining) of the (FIG. 6C) EFP and (FIG. 6D) subcutaneous tissue implanted with graphene-0.5 wt/v % Dex bioscaffolds; Red arrow=bioscaffold; Blue arrows=blood vessels (photographs and H&E staining images). Representative TNF-α staining of the (FIG. 6E) EFP and (FIG. 6F) subcutaneous tissue implanted with graphene-0.5 wt/v % Dex bioscaffolds; Black arrow=bioscaffold; Red arrows=positive (brown) staining. Photo Credit: Mehdi Razavi, Stanford University.

### DETAILED DESCRIPTION

A bioscaffold comprising a graphene matrix for use in cellular therapy is disclosed. In particular, a bioscaffold having a coating of dexamethasone on a three-dimensional graphene matrix is provided, wherein the bioscaffold elutes dexamethasone to reduce inflammatory responses following implantation of the bioscaffold in a subject. Having the dexamethasone released locally in the vicinity of the bioscaffold avoids the systemic side effects from conventional intravenous delivery while allowing the dexamethasone to modulate the inflammatory milieu within the transplantation microenvironment.

Before the graphene bioscaffolds with dexamethasone and methods of using them in cellular therapy and tissue transplantation, are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a cell” includes a plurality of such cells and reference to “the pore” includes reference to one or more pores and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

“Biocompatible,” as used herein, refers to a property of a material that allows for prolonged contact with a tissue in a subject without causing toxicity or significant damage.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

“Active agent” and “drug” are used interchangeably to refer to any chemical compound that can have a therapeutic and/or preventive effect for a disease when suitably administered to a subject.

“Therapeutically effective amount” refers to an amount effective, at dosages and for periods of time necessary, to achieve the desired therapeutic result.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

As used herein, the terms “mesenchymal stromal cells” and “mesenchymal stem cells” are used interchangeably and refer to multipotent cells derived from connective tissue. The terms encompass MSCs derived from various sources including, without limitation, bone marrow, adipose tissue, umbilical cord tissue, molar tooth bud tissue, and amniotic fluid.

“Substantially” as used herein, may be applied to modify any quantitative representation that could permissibly vary without resulting in a change in the basic function to which it is related. For example, a scaffold may have dimensions that deviate somewhat from being flat, if the cell encapsulation and/or tissue graft properties of the scaffold is not materially altered.

“Diameter” as used in reference to a shaped structure (e.g., macropore, micropore, cell aggregate, etc.) refers to a length that is representative of the overall size of the structure. The length may in general be approximated by the diameter of a circle of sphere that circumscribes the structure.

“Flat” as used herein, refers to a shape of an object having wide lateral dimensions compared to a smaller height or depth. The object may have a top surface and bottom surface, each defined by edges extending along the lateral dimensions. The top and bottom surfaces may be substantially parallel to each other.

“Substantially purified” generally refers to isolation of a substance (e.g., compound, polynucleotide, protein, polypeptide, antibody, aptamer) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “diabetes” and “diabetic” refer to a progressive disease of carbohydrate metabolism involving inadequate production or utilization of insulin, frequently characterized by hyperglycemia and glycosuria. The terms “pre-diabetes” and “pre-diabetic” refer to a state wherein a subject does not have the characteristics, symptoms and the like typically observed in diabetes, but does have characteristics, symptoms and the like that, if left untreated, may progress to diabetes. The presence of these conditions may be determined using, for example, either the fasting plasma glucose (FPG) test or the oral glucose tolerance test (OGTT). Both usually require a subject to fast for at least 8 hours prior to initiating the test. In the FPG test, a subject's blood glucose is measured after the conclusion of the fasting; generally, the subject fasts overnight and the blood glucose is measured in the morning before the subject eats. A healthy subject would generally have an FPG concentration between about 90 and about 100 mg/dl, a subject with “pre-diabetes” would generally have an FPG concentration between about 100 and about 125 mg/dl, and a subject with “diabetes” would generally have an FPG level above about 126 mg/dl. In the OGTT, a subject's blood glucose is measured after fasting and again two hours after drinking a glucose-rich beverage. Two hours after consumption of the glucose-rich beverage, a healthy subject generally has a blood glucose concentration below about 140 mg/dl, a pre-diabetic subject generally has a blood glucose concentration about 140 to about 199 mg/dl, and a diabetic subject generally has a blood glucose concentration about 200 mg/dl or above. The term “insulin resistance” as used herein refers to a condition where a normal amount of insulin is unable to produce a normal physiological or molecular response. In some cases, a hyper-physiological amount of insulin, either endogenously produced or exogenously administered, is able to overcome the insulin resistance, in whole or in part, and produce a biologic response.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with hyperglycemia, pre-diabetic, or diabetic) as well as those in which prevention is desired (e.g., those with increased susceptibility to diabetes, those having a genetic predisposition to developing diabetes, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

As used herein, the term “cell viability” refers to a measure of the number of cells that are living or dead, based on a total cell sample. High cell viability, as defined herein, refers to a cell population in which greater than 80% of all cells are viable, preferably greater than 90-95%, and more preferably a population characterized by high cell viability containing more than 97-99% viable cells.

### Graphene Bioscaffold

As discussed above, a bioscaffold comprising a graphene matrix for use in cellular therapy is provided. The bioscaffold is coated with dexamethasone and elutes dexamethasone, which reduces inflammatory responses following implantation of the bioscaffold in a subject. Having the dexamethasone released locally in the vicinity of the bioscaffold avoids the systemic side effects from conventional intravenous delivery while allowing the dexamethasone to modulate the inflammatory milieu within the transplantation microenvironment. Accordingly, bioscaffolds comprising a graphene matrix coated with dexamethasone can be used to avoid transplant rejection and prolong survival of transplanted cells.

The dexamethasone-coated graphene bioscaffolds, described herein, can elute dexamethasone in the vicinity of the transplanted cells for at least 2 weeks. In certain embodiments, the dexamethasone is at a concentration of about 0.25 to about 1 weight/volume percent (w/v %) in the bioscaffold, including any concentration within this range such as 0.25 w/v %, 0.30 w/v %, 0.35 w/v %, 0.4 w/v %, 0.45 w/v %, 0.50 w/v %, 0.55 w/v %, 0.60 w/v %, 0.65 w/v %, 0.70 v, 0.75 w/v %, 0.80 w/v %, 0.85 w/v %, 0.90 w/v %, 0.95 w/v %, or 1.00 w/v %. In certain embodiments, the bioscaffold further comprises a polydopamine nanolayer on the surface of the graphene matrix, wherein the polydopamine nanolayer is functionalized with the dexamethasone.

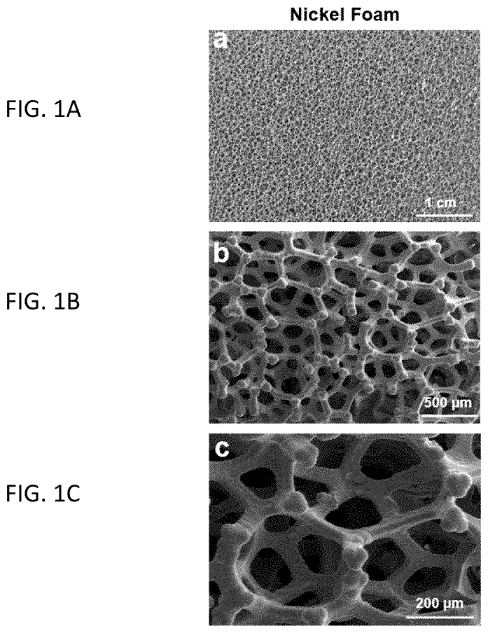

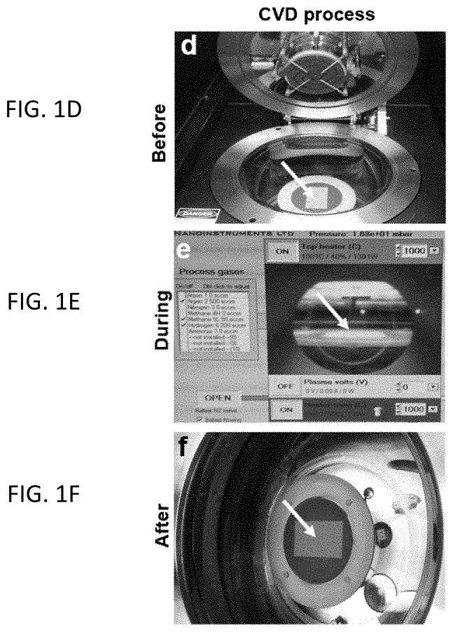

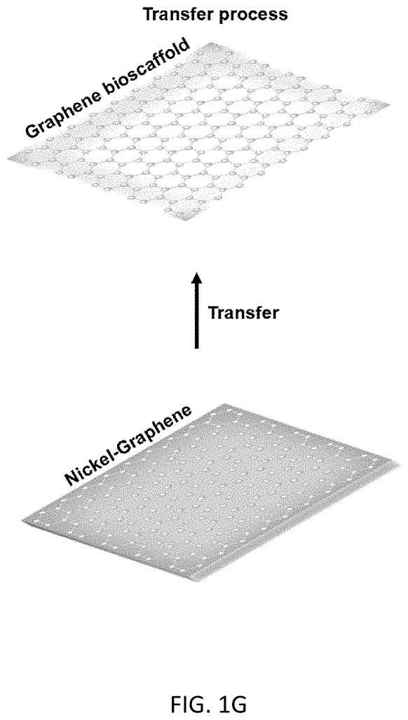

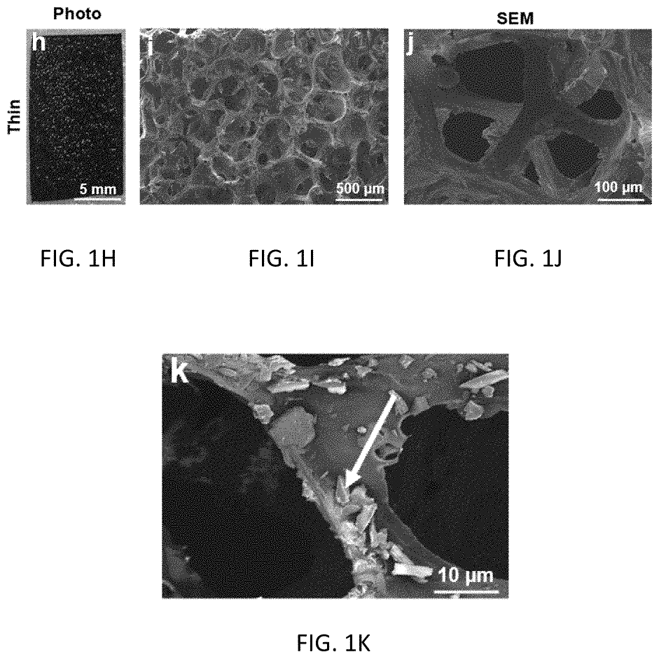

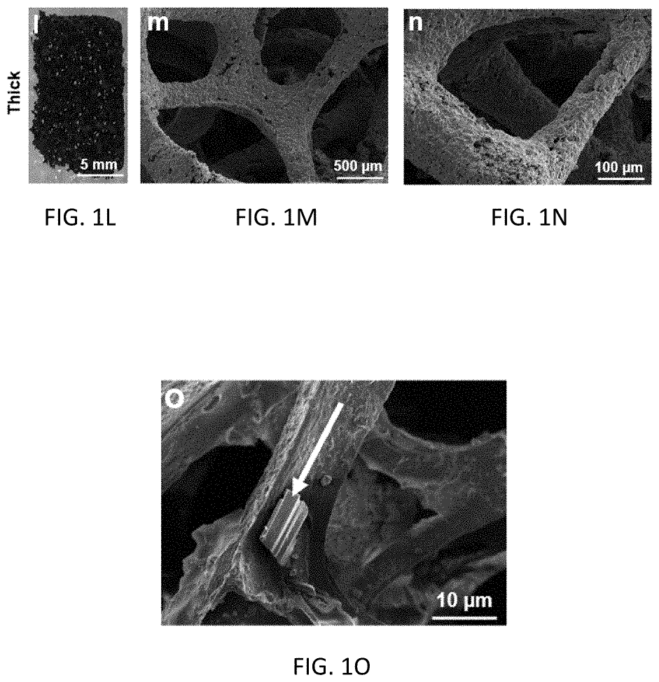

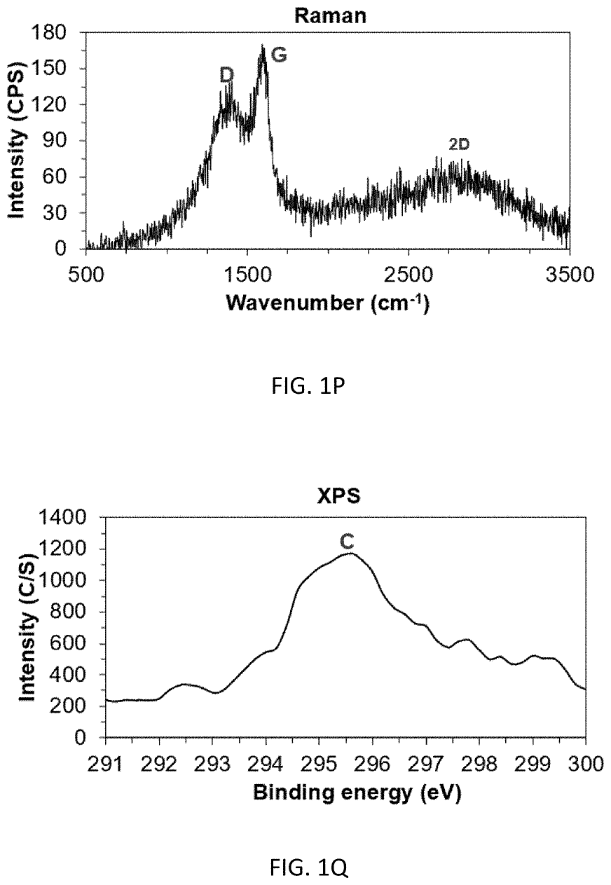

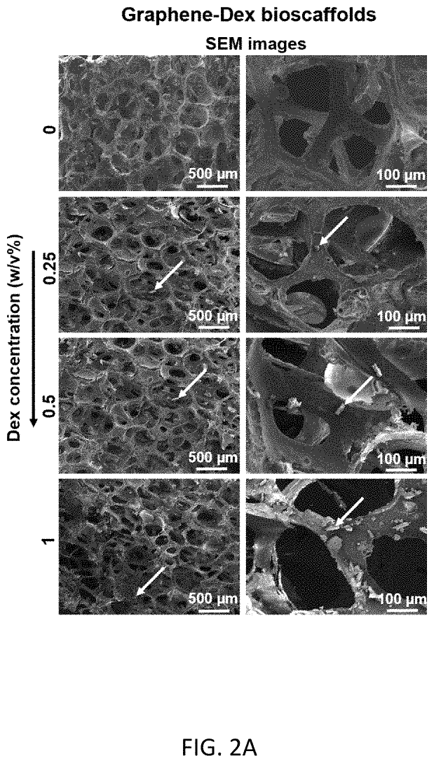

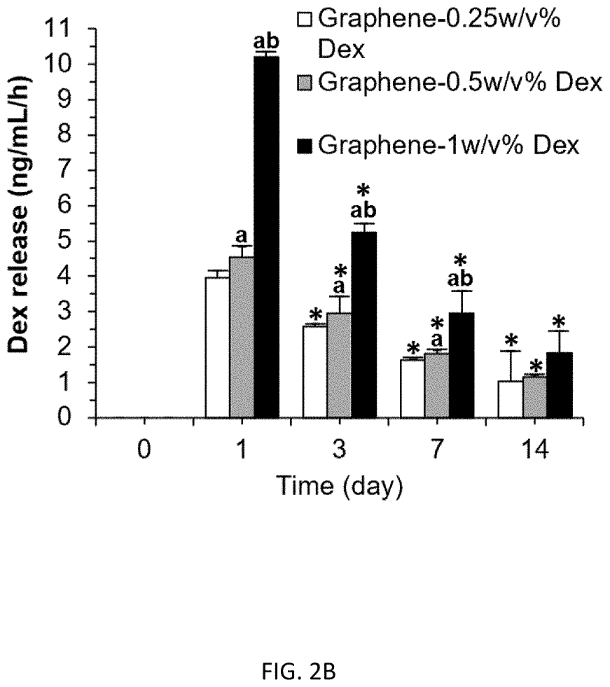

The graphene bioscaffold provides an environment, e.g., microenvironment, for various types of cells, such as therapeutic cells for use in cellular therapy, to attach and grow therein. The bioscaffold structure contains pores, including macropores and micropores (See FIGS. 1I-1K, 1M-1O, and 2A). The macropores generally have an average diameter large enough to accommodate therapeutic cells or cell aggregates.

In some embodiments, the macropores have an average diameter ranging from about 400 μm to 800 μm, including any average diameter within this range, such as 400 μm, 420 μm, 440 μm, 460 μm, 480 μm, 500 μm, 520 μm, 540 μm, 560 μm, 580 μm, 600 μm, 620 μm, 640 μm, 660 μm, 680 μm, 700 μm, 720 μm, 740 μm, 760 μm, 780 μm, or 800 μm, as measured by scanning electron microscopy. In some embodiments, the macropores have a diameter of about 400 μm or more, e.g., about 450 μm or more, about 500 μm or more, about 600 μm or more, about 650 μm or more, about 700 μm or more, including about 750 μm or more, and may have a diameter of about 800 μm or less, e.g., about 700 μm or less, about 600 μm or less, about 500 μm or less, about 475 μm or less, including about 450 μm or less.

In certain embodiments, the micropores have an average diameter ranging from about 100 μm to about 200 μm, as measured by scanning electron microscopy, including any average diameter within this range such as 100 μm, 110 μm, 120 μm, 130 μm, 140 μm, 150 μm, 160 μm, 170 μm, 180 μm, 190 μm, or 200 μm, as measured by scanning electron microscopy. In some embodiments, the micropores have an average diameter of about 200 μm or less, e.g., about 190 μm or less, about 180 μm or less, about 170 μm or less, about 160 μm or less, about 150 μm or less, about 140 μm or less, about 130 μm or less, about 120 μm or less, about 110 μm or less, about 108 μm or less, about 106 μm or less, about 104 μm or less, about 103 μm or less, about 102 μm or less, including about 101 μm or less. In some embodiments, the micropores have an average diameter in the range of about 100 μm to about 200 μm, e.g., about 110 μm to about 190 μm, including about 120.0 μm to about 180 μm.

The bioscaffold has a suitable porosity for supporting growth and/or maintenance of cells encapsulated therein. In some embodiments, the scaffold has a porosity of about 50% or more, e.g., about 55% or more, about 60% or more, about 65% or more, about 70% or more, including about 75% or more, and in some cases, has a bulk porosity of about 95% or less, e.g., about 90% or less, about 85% or less, about 80% or less, including about 75% or less. In some embodiments, the scaffold has a porosity in the range of about 50% to about 95%, e.g., about 55% to about 90%, about 60% to about 85%, including about 65% to about 80%. In some cases, the scaffold has a porosity of about 70% to about 75%. In certain embodiments, the graphene matrix has a porosity ranging from 55% to 95%, including any porosity within this range such as 55%, 57%, 59%, 61%, 63%, 65%, 67%, 69%, 71%, 73%, 75%, 77%, 79%, 81%, 83%, 85%, 87%, 89%, 91%, 93%, or 95%. In some embodiments, the graphene matrix has a porosity of about 80 percent. The porosity may be measured, for example, using computed tomography (CT) scanning (see Examples).

The bioscaffold may have any suitable size or shape for implanting at a physiological site in an individual. In some cases, the bioscaffold has a form factor that is suitable for implanting at a subcutaneous site or in the omentum. In some embodiments, the bioscaffold is substantially flat. The thickness of a substantially flat scaffold may vary, and may be about 0.1 millimeters (mm) or more, e.g., about 0.2 mm or more, about 0.3 mm or more, about 0.5 mm or more, about 0.75 mm or more, about 1.0 mm or more, about 2.0 mm or more, about 3.0 mm or more, about 4.0 mm or more, including about 6.0 mm or more, and in some cases may be about 25 mm or less, e.g., about 20 mm or less, about 15 mm or less, about 10 mm or less, about 8.0 mm or less, about 6.0 mm or less, about 5.0 mm or less, about 4.5 mm or less, about 4.0 mm or less, about 3.5 mm or less, about 3.0 mm or less, about 2.5 mm or less, including about 2.0 mm or less. In some cases, the bioscaffold has a thickness in the range of about 0.1 mm to about 25 mm, e.g., about 0.5 mm to about 20 mm, about 1 mm to about 10 mm, about 2 mm to about 8.0 mm, about 3 mm to about 7.0 mm, about 4 mm to about 6 mm, including about 4.5 mm to about 5.5 mm. In certain embodiments, the bioscaffold has a thickness of from about 0.1 mm to about 25 mm, including any thickness within this range such as 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 8.0 mm, 9.0 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the thickness ranges from about 0.5 mm to about 5 mm.

The bioscaffold may have one or more surfaces with any suitable shape. For example, a substantially flat bioscaffold may have top and bottom surfaces that are in a suitable shape. In some cases, the shape of the surface is circular, square, rectangular, oval, triangular, hexagonal, octagonal, pentagonal, diamond-shaped, parallelogram-shaped, etc. In some cases, the bioscaffold is rectangular (see, Example 1 and FIG. 1G).

The bioscaffold may have any suitable lateral dimensions (e.g., width and/or length, or diameter). In some cases, the bioscaffold has a lateral dimension of about 5 mm or more, e.g., about 6 mm or more, about 7 mm or more, about 8 mm or more, about 9 mm or more, about 1.0 cm or more, about 2.0 cm or more, about 3.0 cm or more, about 4.0 cm or more, including 5 cm or more, and in some cases has a lateral dimension of about 10 cm or less, e.g., about 9.0 cm or less, about 8.0 cm or less, about 7.0 cm or less, about 6.0 cm or less, including about 5.0 cm or less. In some embodiments, the bioscaffold has a lateral dimension in the range of about 1.0 mm to about 10 mm, e.g., about 1.0 mm to about 9.0 mm, about 2.0 mm to about 8.0 mm, about 3.0 mm to about 7.0 mm, including about 4.0 mm to about 6.0 mm.

A bioscaffold having therapeutic cells encapsulated therein may provide a carrier for transplanting cells into a physiological site by implanting the bioscaffold at the site. Also provided herein is a tissue graft that includes the bioscaffold and therapeutic cells encapsulated within the macropores of the graphene matrix. In some cases, where the cells grow as aggregates (where two or more cells are attached to one another) when grown in a conventional culture environment (e.g., grown on a two-dimensional culture dish or flask surface), the macropores have an average diameter that approximates the average size of the cell aggregates. In other cases, individual cells are distributed in the macropores throughout the bioscaffold.

In some cases, the bioscaffold may provide for maintenance or growth of the cells cultured therein for a time period (e.g., one day or more, two days or more, 3 days or more, 4 days or more, 5 days or more, 6 days or more, 8 days or more, 10 days or more, 1 week or more, 2 weeks or more, 3 weeks or more) in in vitro culture. Thus, cells encapsulated in the bioscaffold may at least maintain the same number of cells, or may expand by two times or more, e.g., 3 times or more, 4 times or more, 5 times or more, 10 times or more, 20 times or more 50 times or more, including 100 times or more in number after a time period in culture as compared to the number of cells initially seeded on the scaffold. In some cases, cells encapsulated in the bioscaffold expands by a range of 2-fold to 1000-fold, e.g., 2-fold to 100-fold, 2-fold to 50-fold, including 3-fold to 20-fold in number after the time period in culture.

The bioscaffold, with or without cells encapsulated therein, may promote vascularization when the bioscaffold is implanted into a physiological site (e.g., kidney, liver, omentum, peritoneum, abdomen, or submuscular or subcutaneous tissue) of an individual. The implantation site may be vascularized in 60 days or less, e.g., 40 days or less, 30 days or less, 20 days or less, 10 days or less, including 5 days or less after implantation of the bioscaffold, with or without cells encapsulated therein. In some cases, the implantation site is vascularized in 5 days to 60 days, e.g., 5 days to 40 days, including 10 days to 30 days after implantation of the bioscaffold, with or without cells encapsulated therein. In some cases, the vascularization occurs with little or no fibrosis around the bioscaffold, at the interface between the host tissue and the bioscaffold.

A tissue graft of the present disclosure containing therapeutic cells may maintain the cells in a functional state suitable for providing a therapeutic effect (e.g., insulin secretion by beta-cells) when implanted into a physiological site (e.g., kidney, liver, omentum, peritoneum, abdomen, or submuscular or subcutaneous tissue) of an individual. The therapeutic cells may maintain responsiveness to physiological cues (e.g., blood glucose level) at the implantation site.

Without wishing to be bound by theory, it is thought that the porosity (i.e., the macro- and micro-porosity) of the bioscaffold presents to the encapsulated cells a microenvironment that provides desirable nutrient transport and vascular integration to grow and maintain the cells in a functional state. The dexamethasone coating the graphene matrix of the bioscaffold has an anti-inflammatory effect that protects the cells from the host mediated foreign body response (FBR) following implantation and prolongs survival of the cells.

Alternatively or additionally, a bioscaffold may be used as a carrier for transplanting extracellular vesicles into a physiological site by implanting a bioscaffold containing extracellular vesicles at the site. Extracellular vesicles include, without limitation, exosomes, ectosomes, microvesicles, or other microparticles derived from the plasma membrane of a cell. In some cases, the extracellular vesicles are derived from a stem cell (e.g., embryonic stem cell, adult stem cell, induced pluripotent stem cell). In some cases, the extracellular vesicles are derived from a mesenchymal stem cell.

### Tissue Grafts

Also provided herein is a tissue graft that includes a bioscaffold comprising a graphene matrix coated with dexamethasone, as described above, and therapeutic cells encapsulated within the macropores of the graphene matrix. The therapeutic cells are, in some cases, stably encapsulated within the scaffold such that the cells remain in the bioscaffold when the tissue graft is implanted at an implantation site (e.g., a subcutaneous, intra-abdominal/intraperitoneal, or submuscular site) of an individual. In some cases, about 20% or less, e.g., about 15% or less, about 10% or less, about 5% or less, about 2% or less, about 1% or less, about 0.1% or less, about 0.01% or less, including about 0.001% or less of the total number of therapeutic cells encapsulated in the scaffold may exit the scaffold when implanted at an implantation site of an individual.

In some cases, the therapeutic cells can migrate out of the scaffold when the tissue graft is implanted at an implantation site of an individual. The extent to and/or rate at which therapeutic cells exit the scaffold when the tissue graft is implanted at an implantation site of an individual may vary, and may depend on a variety of controllable factors, such as the size of the pores (macropores and/or micropores), the size of the pores (macropores and/or micropores) relative to the size of the cells or cell aggregates encapsulated in the scaffold, the density of the graphene matrix, the porosity of the graphene matrix, surface modification of the bioscaffold, etc.

The tissue graft may have encapsulated therein any suitable amount of the therapeutic cells. The amount of cells may depend on a variety of factors, such as the function provided by the therapeutic cells, the size of the tissue graft, the length of time the tissue graft is to be implanted, the condition to be treated by the tissue graft and/or the desired therapeutic outcome. In some cases, the tissue graft includes at least 10⁵ cells, e.g., at least 10⁶ cells, at least 10⁷ cells, at least 10⁸ cells, at least 10⁹ cells, at least 10¹⁰ cells, or more cells.

In some embodiments, individual cells are distributed in the macropores throughout the graphene matrix, which may improve cell survival and function by preventing clumping of cells. In other embodiments, the therapeutic cells are aggregating cells. Aggregating cells may be cells that, when grown on the surface of a culture dish or in suspension, attach to one another to form clumps (i.e., aggregates) of two or more cells, e.g., 10 or more cells, 100 or more cells, 1,000 or more cells, including 10,000 or more cells. The aggregate of cells may also be attached to a solid support (e.g., the culture dish surface) or may be free-floating in the medium. The aggregate of cells may be any suitable shape, and in some cases, may be spherical or oval. The size of the aggregate may be any suitable size. In some cases, the cell aggregate that forms in a conventional culture condition (e.g., in suspension, or on a two-dimensional surface) has an average diameter that approximates the average diameter of the macropores of the present bioscaffold. Thus, in some cases, the therapeutic cells may form, in a conventional culture condition, cell aggregates having an average diameter that is within about 50%, e.g., within about 40%, within about 30%, within about 20%, within about 10%, including within about 5% of the average diameter of the macropores of the bioscaffold of the present tissue graft. In some embodiments, the therapeutic cells may form, in a conventional culture condition, cell aggregates having an average diameter of about 50 μm or more, e.g., about 75 μm or more, about 100 μm or more, including about 125 μm or more, and in some cases, an average diameter of about 300 μm or less, e.g., about 275 μm or less, about 250 μm or less, about 225 μm or less, including about 200 μm or less. In some cases, the therapeutic cells may form, in a conventional culture condition, cell aggregates having an average diameter in the range of about 50 μm to about 300 μm, e.g., about 75 μm to about 275 μm, about 75 μm to about 250 μm, about 100 μm to about 225 μm, including about 100 μm to about 200 μm.

The aggregate of cells may be a collection of a substantially pure population of cells, or may be a collection of a plurality of types, e.g., two more types, three or more types, four or more types, including 5 or more types, of cells. In some cases, the aggregate of cells is stem cell-derived. In some cases, the aggregate of cells is an embryoid body that includes pluripotent stem cells and/or cells differentiated therefrom.

In some embodiments, the therapeutic cells include cells that secrete a biological agent, e.g., a signaling molecule, a hormone, a growth factor, a cytokine, a chemokine, an enzyme, an antibody, etc. In some cases, the therapeutic cells include cells (e.g., immune cells, such as cytotoxic T lymphocytes) that interact with targets at or in the vicinity in the host tissue in which the tissue graft is implanted. In some cases, the therapeutic cells include cells whose activity is conditional, e.g., cells that modulate their function based on the physiological state of the host, such as glucose level in the blood and/or the environment of the host tissue. The therapeutic cell may be a type of cell that specifically possesses the functional activity by virtue of its cell type (e.g., by differentiating or having differentiated into a cell type that exhibits the functional activity), or may be genetically modified to exhibit the functional activity that was not exhibited by the cell before being genetically modified.

Exemplary therapeutic molecules that can be secreted by a therapeutic cell include, without limitation, insulin, human growth hormone, thyroxine, glucagon-like peptide-1 (GLP-1), GLP-1 (7-37), GLP-1 (7-36), and like GLP-1 receptor agonist polypeptides, GLP-2, interleukins 1 to 33 (e.g., IL-1, IL-2, IL-3, IL-4, IL-5, IL-6, IL-7, IL-8, IL-9, IL-10, IL-11, IL-12, IL-13, IL-17, IL-18, IL-21, IL-22, IL-27, IL-33), interferon (α, β, γ), GM-CSF, G-CSF, M-CSF, SCF, FAS ligands, TRAIL, leptin, adiponectin, blood coagulation factor VIII/blood coagulation factor IX, von Willebrand factor, glucocerebrosidase, lipoprotein lipase (LPL), lecithin-cholesterol acyltransferase (LCAT), erythropoietin, apoA-I, albumin, atrial natriuretic peptide (ANP), luteinizing hormone releasing hormone (LHRH), angiostatin/endostatin, endogenous opioid peptides (enkephalins, endorphins, etc.), calcitonin/bone morphogenetic protein (BMP), pancreatic secretory trypsin inhibitors, catalase, superoxide dismutase, anti-TNF-α antibody, soluble IL-6 receptor, IL-1 receptor antagonist, α2 antitrypsin, etc.

The therapeutic cells may be any suitable type of cell for transplanting to an individual in need. The therapeutic cells may be stem cells, progenitor cells, or mature cells. The cells may be autologous, allogeneic, xenogeneic or genetically-modified.

In some cases, the therapeutic cells are stem cell-derived cells. Stem cells of interest include, without limitation, hematopoietic stem cells, embryonic stem cells, adult stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, induced pluripotent stem cells; and the like; as well as differentiated cells that can be cultured in vitro and used in a therapeutic regimen, where such cells include, but are not limited to, keratinocytes, adipocytes, cardiomyocytes, neurons, osteoblasts, pancreatic islet cells, retinal cells, and the like. The cell that is used will depend in part on the nature of the disorder or condition to be treated.

Suitable human embryonic stem (ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIII) code, followed in parentheses by the provider code.

Hematopoietic stem cells (HSCs) are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSCs) can be obtained from connective tissue including, without limitation, bone marrow, placenta, umbilical cord blood, adipose tissue, muscle, corneal stroma, dental pulp of deciduous baby teeth, molar tooth bud tissue, and amniotic fluid. MSCs can differentiate to form muscle, bone, cartilage, fat, marrow stroma, and tendon. Methods of isolating MSCs are known in the art; and any known method can be used to obtain MSCs. The MSCs may be obtained directly from the patient to be treated, a donor, a culture of cells from a donor, or from established cell culture lines. In some embodiments, the graphene scaffold encapsulates MSCs and/or MSC-derived extracellular vesicles. MSC-derived extracellular vesicles include, without limitation, exosomes, ectosomes, microvesicles, or other microparticles derived from the plasma membrane of MSCs.

An induced pluripotent stem (iPS) cell is a pluripotent stem cell induced from a somatic cell, e.g., a differentiated somatic cell. iPS cells are capable of self-renewal and differentiation into cell fate-committed stem cells, including neural stem cells, as well as various types of mature cells. iPS cells can be generated from somatic cells, including skin fibroblasts, using, e.g., known methods. iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and K1 f4. In some embodiments, somatic cells are genetically modified with one or more expression constructs comprising nucleotide sequences encoding Oct-4, Sox2, Nanog, and LIN28. Methods of generating iPS are known in the art, and any such method can be used to generate iPS.

In some cases, the therapeutic cells are lymphocytes, such as CD4+ and/or CD8+T lymphocytes, or B lymphocytes. In some embodiments, the therapeutic cells are cytotoxic T lymphocytes. In some embodiments, the lymphocytes are genetically modified lymphocytes, e.g., chimeric antigen receptor (CAR) T lymphocytes. The lymphocytes, e.g., cytotoxic T lymphocytes, may specifically recognize an antigen that is associated with a disease, e.g., cancer or tumor, that is to be treated with the tissue graft.

In some embodiments, the therapeutic cells include insulin-secreting cells. The insulin-secreting cells may be any suitable type of insulin-secreting cell. In some cases, the insulin-secreting cells are a type of cell that secretes insulin (e.g., pancreatic β islet cells, or β-like cells). In some cases, the insulin-secreting cells are primary β islet cells (e.g., mature β islet cells isolated from a pancreas). In some cases, the insulin-secreting cells are β cells, or β-like cells that are derived in vitro from immature cells, precursor cells, progenitor cells, or stem cells. The insulin-secreting cells may be derived from (i.e., obtained by differentiating) stem and/or progenitor cells such as hepatocytes (e.g., transdifferentiated hepatocytes), acinar cells, pancreatic duct cells, stem cells, embryonic stem cells (ES), partially differentiated stem cells, non-pluripotent stem cells, pluripotent stem cells, induced pluripotent stem cells (iPS cells), etc. Suitable insulin-secreting cells and methods of generating the same are described in, e.g., US20030082810; US20120141436; and Raikwar et al. (PLoS One. 2015 Jan. 28;10(1):e0116582), each of which are incorporated herein by reference.

The insulin-secreting cells may produce (e.g., secrete) insulin at a rate independent of the ambient/extracellular glucose concentration (e.g., the concentration of glucose in the host tissue in which the tissue graft is implanted), or may produce (e.g., secrete) insulin at a rate that depends on the ambient/extracellular glucose concentration. In some cases, the insulin-secreting cells constitutively secrete insulin. In some embodiments, the insulin-secreting cells increase insulin secretion when the ambient/extracellular glucose concentration increases, and decreases insulin secretion when the ambient/extracellular glucose concentration decreases.

The tissue graft may include a suitable coating on the bioscaffold (e.g., on the surface of the macropores and/or micropores of the scaffold) to promote encapsulation of the therapeutic cells. The coating may include a biological coating (e.g., extracellular matrix proteins) and/or may include a synthetic coating (such as described in US20070032882, which is incorporated herein by reference). A suitable biological coating includes extracellular matrix proteins, such as, without limitation, collagen, fibronectin, vitronectin, laminin, heparan sulfate, proteoglycan, glycosaminoglycan, chondroitin sulfate, hyaluronan, dermatan sulfate, keratin sulfate, elastin, and combinations thereof.

In some embodiments, the present tissue graft includes one or more active agents adsorbed or absorbed within the bioscaffold, wherein the bioscaffold is configured to deliver the one or more active agents to a site of implantation. In some embodiments, the active agent is an immunosuppressant, such as, but not limited to cyclosporine and tacrolimus. In some cases, the active agent is an inhibitor of the mammalian target of rapamycin (mTOR), such as, without limitation, rapamycin and analogs thereof (e.g., sirolimus, temsirolimus, everolimus, deforolimus, etc.). The mTOR inhibitor may be used as an immunosuppressant, or may be an anticancer agent. In some cases, the active agent is a binding agent, such as an antibody, or an antigen binding fragment thereof. The antibody may be any suitable antibody that specifically binds to an antigen expressed by a therapeutic cell of interest for encapsulating in the present scaffolds. Suitable antigens include, without limitation, CD3, CD28, CD137, CTLA-4, TNF, IL-6, IL-12, PD-1, PD-L1, TIM3, LAGS, IL-2Ralpha, IL-23, IL-6R, CD25, IL-17, IL-1, CD4, CD8, LFA-1, IL-22, and IL-20.

Other suitable active agents according to embodiments of the present disclosure may include but are not limited to interferon, interleukin, erythropoietin, granulocyte-colony stimulating factor (GCSF), stem cell factor (SCI), leptin (OB protein), interferon (alpha, beta, gamma), antibiotics such as vancomycin, gentamicin ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, glial derived neurotrophic factor (GDNF), neurotrophic factor 3 (NT3), fibroblast growth factor (FGF), transforming growth factor (TGF), platelet transforming growth factor, milk growth factor, endothelial growth factors, endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4- IBBR), TRAIL (TNF-related apoptosis inducing ligand), artemin (GFRalpha3-RET ligand), BCA-1 (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, thrombopoietin, megakaryocyte derived growth factor (MDGF), keratinocyte growth factor (KGF), platelet-derived growth factor (PDGF), ciliary neurotrophic factor (CNTF), neurotrophin 4 (NT4), granulocyte colony-stimulating factor (GCSF), macrophage colony-stimulating factor (mCSF), bone morphogenetic protein 2 (BMP2), BRAK, C-10, Cardiotrophin 1 (CTI), CCR8, anti-inflammatory: paracetamol, salsalate, diflunisal, mefenamic acid, diclofenac, piroxicam, ketoprofen, dipyrone, acetylsalicylic acid, anti-cancer drugs such as aliteretinoin, altertamine, anastrozole, azathioprine, bicalutarnide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, antivirus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetaminophen, analgesics, antitumor preparations, anti-cancer preparations, anti-proliferative preparations or pro-apoptotic preparations, among other types of active agents.

In some embodiments, the one or more absorbed active agents is a compound selected from the group consisting of chemotactic agents, cell attachment mediators, integrin binding sequences, epidermal growth factor (EGF), hepatocyte growth factor (HGF), vascular endothelial growth factors (VEGF), fibroblast growth factors, platelet derived growth factors (PDGF), insulin-like growth factor, transforming growth factors (TGF), parathyroid hormone, parathyroid hormone related peptide, bone morphogenetic proteins (BMP), BMP-2, BMP-4, BMP-6, BMP-7, BMP-12, BMP-13, BMP-14, transcription factors, growth differentiation factor (GDF), GDF5, GDF6, GDF8, recombinant human growth factors, cartilage-derived morphogenetic proteins (CDMP), CDMP-1, CDMP-2 and CDMP-3.

Methods of Transplanting Cells into an Individual

Also provided herein is a method of transplanting cells into an individual, using a graphene bioscaffold, as described herein, e.g., to treat a disease. The method may include implanting (e.g., surgically implanting) a graphene bioscaffold containing the therapeutic cells, encapsulated in the pores (e.g., macropores) of the bioscaffold, into an implantation site of a host individual. The host individual may be suffering from a condition, e.g., a disease, that may be treated by providing the therapeutic cells to the individual. In some cases, the disease is diabetes (type 1 or type 2). In some cases, the individual has pre-diabetes, or hyperglycemia. In some cases, the disease is cancer (e.g., breast cancer, prostate cancer, brain cancer, skin cancer, lung cancer, liver cancer, colorectal cancer, etc.). The therapeutic cells may be any suitable therapeutic cells, as described above, and the type of therapeutic cells may depend on the disease to be treated.

The implantation site may be any suitable location (e.g., surgically accessible location) in the individual. In some cases, the implantation site is in a kidney, liver, omentum, peritoneum, abdomen, or submuscular or subcutaneous tissue. In some cases, the implantation site is at or in the vicinity of a tissue that is affected by the disease (e.g., a tissue with a solid tumor).

The graphene bioscaffold elutes dexamethasone at the implantation site to protect the therapeutic cells from the host mediated foreign body response (FBR) following its implantation. The period of time may be 1 week or more, 2 weeks or more, 3 weeks or more, 4 weeks or more, 50 days or less, e.g., 40 days or less, 35 days or less, 30 days or less, including 20 days or less. In some cases, the period is in the range of 14 days to 50 days, e.g., 14 days to 40 days, 14 days to 30 days, including 14 days to 20 days. In some cases, implanting the tissue graft may be performed in conjunction with another therapy, such as another surgical operation and/or administration of a drug. In some cases, the tissue graft is implanted at an implantation site after a surgical operation, e.g., to remove a tumor or malignant tissue from the implantation site.

Also provided herein is a method of regulating blood glucose levels in an individual using a graphene bioscaffold, as described herein. The present method may include implanting a graphene bioscaffold comprising insulin-secreting cells encapsulated in its pores (e.g., macropores), as described herein, into an implantation site (e.g., site in a kidney, liver, omentum, peritoneum, abdomen, or submuscular or subcutaneous tissue) of a host individual, to maintain normoglycemia in the individual. The individual may be suffering from dysregulation of blood glucose, and may have, e.g., type 1 or type 2 diabetes, pre-diabetes, or hyperglycemia. The insulin-secreting cells may be any suitable insulin-secreting cells, as described above. The tissue graft may include any suitable number of cells, as described above, and in some cases includes 10⁵ to 10⁹ cells, e.g., 10⁶ to 10⁸ cells. The present method may further include preparing the tissue graft by culturing the insulin-secreting cells on the bioscaffold, as described herein, to encapsulate the insulin-secreting cells in the macropores of the bioscaffold.

A medical practitioner may locate the site for implantation of a bioscaffold comprising therapeutic cells or a tissue graft, for example, by medical imaging (e.g. ultrasound, radiography, or MRI). In some embodiments, a contrast agent is included in the composition comprising the therapeutic cells to allow confirmation of the location of the cells by medical imaging after implantation. In some embodiments, the contrast agent is a microbubble (e.g., for use in ultrasound) or a radiopaque contrast agent (e.g., for use in radiography). The contrast agent may be contained in the same composition as the therapeutic cells or in a different composition and used prior to or after implantation of the bioscaffold.

### Utility

The graphene bioscaffolds and tissue grafts find many uses where it is desirable to transplant a population of therapeutic cells into an individual to treat a condition, e.g., a disease. As described herein, a variety of types of therapeutic cells (e.g., cells that secrete a hormone or enzyme, cytotoxic cells targeting a tumor, etc.) can be loaded into the porous scaffold, which elutes dexamethasone at the implantation site to protect the therapeutic cells from the host-mediated foreign body response following its implantation. The scaffold promotes vascularization, and minimizes inflammation and foreign body responses, such as fibrosis at the site of implantation.

In some cases, where the therapeutic cells substantially remain in the tissue graft when implanted in the host tissue, the therapeutic cells may be removed, if necessary, by removing the entire tissue graft. In such cases, the graphene scaffold may be designed (e.g., by providing an appropriate porosity) to retain the therapeutic cells in the scaffold over the desired duration of time.

### Kits

Also provided herein is a kit comprising a bioscaffold comprising a graphene matrix coated with dexamethasone that can be used in performing methods of the present disclosure. The kit may also include a packaging that includes a compartment, e.g., a sterile compartment, for holding the bioscaffold. The packaging may be any suitable packaging for holding the bioscaffold. Examples of packaging and methods of packaging are described in, e.g., U.S. Pat. Nos. 3,755,042, 4,482,053, 4,750,619; U.S. App. Pub. Nos. 20050268573, 20100133133, each of which are incorporated herein by reference.

In some cases, the present kit further contains cells, e.g., therapeutic cells, or a precursor thereof, suitable for forming a tissue graft, as described herein. In some embodiments, the cells are encapsulated within the pores (e.g., macropores) of the bioscaffold, thereby forming a tissue graft.

In some cases, the bioscaffold or tissue graft further comprises an active agent. The different components of the kit may be provided in separate containers, as appropriate.

The kit may also include instructions for using the graphene bioscaffold and/or tissue graft. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, digital versatile disc (DVD), flash drive, Blue-ray Disc™ etc. In yet other embodiments, the actual instructions are not present in the kit, but methods for obtaining the instructions from a remote source, e.g. via the internet, are provided. An example of this embodiment is a kit that includes a web address where the instructions can be viewed and/or from which the instructions can be downloaded. As with the instructions, the methods for obtaining the instructions are recorded on a suitable substrate.

In certain embodiments, the kit comprises a graphene bioscaffold comprising insulin-secreting cells (e.g., pancreatic beta cells or islets from a donor, or insulin-secreting cells derived from stem cells or pancreatic progenitor cells), as described herein, and instructions for treating diabetes or hyperglycemia.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-53 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A bioscaffold comprising:
a) a three-dimensional graphene matrix, wherein the graphene matrix comprises a plurality of macropores and micropores; and
b) a coating comprising dexamethasone.

2. The bioscaffold of aspect 1, further comprising a polydopamine nanolayer on the surface of the graphene matrix, wherein the polydopamine nanolayer is functionalized with the dexamethasone.

3. The bioscaffold of aspect 1, wherein the graphene matrix has a porosity ranging from 55 percent to 95 percent.

4. The bioscaffold of aspect 3, wherein the graphene matrix has a porosity of about 80 percent.

5. The bioscaffold of aspect 1, wherein the macropores have an average diameter ranging from about 400 μm to about 800 μm as measured by scanning electron microscopy.

6. The bioscaffold of aspect 1, wherein the micropores have an average diameter of 200 μm or less as measured by scanning electron microscopy.

7. The bioscaffold of aspect 6, wherein the micropores have an average diameter ranging from about 100 μm to about 200 μm as measured by scanning electron microscopy.

8. The bioscaffold of aspect 1, wherein the dexamethasone is at a concentration of about 0.25 to about 1 weight/volume percent (w/v %) in the bioscaffold.

9. The bioscaffold of aspect 1, wherein the bioscaffold has a thickness of from about 0.1 mm to about 25 mm.

10. The bioscaffold of aspect 9, wherein the thickness ranges from about 0.5 mm to about 5 mm.

11. The bioscaffold of aspect 1, further comprising one or more drugs, growth factors, angiogenic agents, cytokines, or extracellular matrix components, or a combination thereof.

12. The bioscaffold of aspect 1, wherein the bioscaffold further comprises therapeutic cells or extracellular vesicles, wherein the therapeutic cells or extracellular vesicles are contained in the macropores.

13. The bioscaffold of aspect 12, wherein the therapeutic cells are stem cells, progenitor cells, or mature cells.

14. The bioscaffold of aspect 13, wherein the stem cells are induced-pluripotent stem cells or adult stem cells.

15. The bioscaffold of aspect 13, wherein the stem cells are mesenchymal stem cells.

16. The bioscaffold of aspect 15, wherein the mesenchymal stem cells are adipose tissue-derived mesenchymal stem cells.

17. The bioscaffold of aspect 12, wherein the therapeutic cells secrete a cytokine, a chemokine, an antibody, an enzyme, a growth factor, or a hormone.

18. The bioscaffold of aspect 17, wherein the therapeutic cells are endocrine cells, exocrine cells, stem cells, or lymphocytes.

19. The bioscaffold of aspect 12, wherein the therapeutic cells are genetically modified cells.

20. The bioscaffold of aspect 12, wherein the therapeutic cells are insulin-secreting cells.

21. The bioscaffold of aspect 20, wherein the insulin-secreting cells are pancreatic beta cells or islets obtained from a donor.

22. The bioscaffold of aspect 20, wherein the insulin-secreting cells are derived from stem cells or pancreatic progenitor cells.

23. The bioscaffold of aspect 20, wherein the therapeutic cells further comprise stem cells.

24. The bioscaffold of aspect 23, wherein the stem cells are mesenchymal stem cells.

25. The bioscaffold of aspect 12, wherein the extracellular vesicles are exosomes, ectosomes, microvesicles, or microparticles derived from a plasma membrane of a cell.

26. The bioscaffold of aspect 12, wherein the extracellular vesicles are derived from mesenchymal stem cells.

27. A method of treating a subject for type 1 diabetes, the method comprising implanting the bioscaffold of aspect 20 in the subject at an implantation site.

28. The method of aspect 27, wherein the bioscaffold releases dexamethasone for at least 2 weeks at the implantation site in vivo.

29. The method of aspect 27, wherein the insulin-secreting cells are autologous, allogeneic, or xenogeneic pancreatic beta cells or islets.

30. The method of aspect 27, wherein the insulin-secreting cells are derived from stem cells or pancreatic progenitor cells.

31. The method of aspect 27, wherein the implantation site is in a kidney, liver, omentum, peritoneum, abdomen, submuscular tissue, or subcutaneous tissue of the subject.

32. A tissue graft comprising:
a) the bioscaffold of aspect 1; and
b) a plurality of therapeutic cells encapsulated within the macropores of the graphene matrix.

33. A method of producing a tissue graft, the method comprising:
a) depositing a plurality of therapeutic cells on the bioscaffold of aspect 1; and
b) culturing the deposited therapeutic cells under conditions wherein an effective amount of the therapeutic cells is encapsulated in the macropores of the graphene matrix.

34. The method of aspect 33, wherein the therapeutic cells are insulin-secreting cells.

35. The method of aspect 34, wherein the insulin-secreting cells are pancreatic beta cells or islets obtained from a donor.

36. The method of aspect 34, wherein the insulin-secreting cells are derived from stem cells or pancreatic progenitor cells.

37. The method of aspect 34, wherein the therapeutic cells further comprise stem cells.

38. The method of aspect 37, wherein the stem cells are mesenchymal stem cells.

39. A method for making a graphene bioscaffold of aspect 2, the method comprising:
a) fabricating a three-dimensional graphene matrix using template-directed chemical vapor deposition;
b) coating the surface of the graphene matrix with polydopamine; and
c) functionalizing the polydopamine coating with dexamethasone.

40. The method of aspect 39, further comprising depositing therapeutic cells or extracellular vesicles on the bioscaffold.

41. The method of aspect 40, wherein the therapeutic cells are insulin-secreting cells.

42. The method of aspect 41, wherein the insulin-secreting cells are autologous, allogeneic, or xenogeneic pancreatic beta cells or islets.

43. The method of aspect 41, wherein the insulin-secreting cells are pancreatic beta cells or islets obtained from a donor.

44. The method of aspect 41, wherein the insulin-secreting cells are derived from stem cells or pancreatic progenitor cells.

45. The method of aspect 41, wherein the therapeutic cells further comprise stem cells.

46. The method of aspect 45, wherein the stem cells are mesenchymal stem cells.

47. The method of aspect 39, further comprising adding one or more drugs, growth factors, angiogenic agents, cytokines, or extracellular matrix components, or a combination thereof to the bioscaffold.

48. A method of regulating blood glucose levels in a subject, the method comprising implanting the bioscaffold of aspect 20 in the subject at an implantation site.

49. The method of aspect 48, wherein the insulin-secreting cells are autologous, allogeneic, or xenogeneic pancreatic beta cells or islets.

50. The method of aspect 48, wherein the insulin-secreting cells are pancreatic beta cells or islets obtained from a donor.

51. The method of aspect 48, wherein the insulin-secreting cells are derived from stem cells or pancreatic progenitor cells.

52. The method of aspect 48, wherein the implantation site is in a kidney, liver, omentum, peritoneum, abdomen, submuscular tissue, or subcutaneous tissue of the subject.

53. The method of aspect 48, wherein the subject has hyperglycemia or type 1 diabetes.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Localized Drug Delivery Graphene Bioscaffolds for Co-Transplantation of Islets and Mesenchymal Stem Cells

### Introduction

Islet transplantation is a β cell replacement therapy used to treat diabetic patients who lack the ability to secrete insulin (1). Currently, there are 2 types of islet transplants performed: (i) autologous islet transplantation (an FDA approved and reimbursable procedure which is rapidly becoming the “standard of care” for patients who require a total pancreatectomy for the treatment of chronic pancreatitis with intractable abdominal pain) and (ii) allogeneic islet transplantation (currently performed in many countries for patients with Type 1 diabetes (T1 D) who have poor glycemic control and suffer from hypoglycemia unawareness). In both autologous and allogenic islet transplantation, up to 60% of islets are immediately lost following transplantation; a main reason for this is due to inflammation from the instant blood mediated inflammatory reaction (IBMIR) (2). One strategy to prevent the IBMIR is to not deliver islets into the liver via the portal vein, but instead implant them using a biocompatible three-dimensional (3D) bioscaffold (3) at extravascular sites like the omentum or subcutaneous space (4, 5).

Bioscaffolds are rapidly gaining attraction for islet transplantation (6-12) given that they contain pores that can accommodate islets while simultaneously offering a unique interface that can be modulated to solve several problems currently faced by islets in the immediate post-transplantation period (such as inflammation) (13). Bioscaffolds have predominantly been made from synthetic polymers, including polydimethylsiloxane (PDMS) (7), poly(lactide-co-glycolide) (6), poly(D,L-lactide-co-e-caprolactone) (8), poly(ethylene oxide terephtalate)/poly(butylene terephthalate) block copolymer (9) and heparin-binding peptide amphiphiles (10). Despite promising pre-clinical data in providing immune-isolation of islets (6-11), the clinical translation of many of these platforms has been limited due to (i) their inability to be reproducibly scaled, (ii) adverse biomaterial-soft tissue interface reactions that adversely affects islets; especially for synthetic materials (14) and (iii) difficulties in functionalizing the surface of certain bioscaffolds. However, one rapidly emerging material which can address many of these issues is graphene; this one-atom-thick fabric of carbon not only has extremely high mechanical strength with excellent flexibility (15), but it also has excellent electronic and thermal conductivities making it an attractive material for use in biological applications. Furthermore, its high chemical purity (16) allows for bioscaffolds to be constructed with low lattice defects (17) and excellent biocompatibility (18).

While graphene will not induce an intense inflammatory response like synthetic materials, there will still be some degree of a host mediated foreign body response (FBR) following its implantation which is likely to adversely affect islets (19). However, graphene can be easily functionalized (20) with anti-inflammatory drugs, like dexamethasone (Dex), thereby enabling it to act as a localized anti-inflammatory drug delivery platform. This approach will avoid the systemic side effects from conventional intravenous delivery of Dex, while concurrently enabling it to precisely modulate the inflammatory milieu within the transplantation microenvironment. Dex is an immunosuppressive glucocorticoid that significantly reduces the activation of inflammatory pathways and has been shown to polarize monocytes toward the anti-inflammatory (M2) phenotype, while still retaining their migratory function (21). However, the dose of Dex needs to be carefully tailored, as elevated levels can severely impair cell mobility resulting in compromised engraftment and vascularization (22), as well as the glucose responsiveness of β cells in transplanted islets (23).

In addition to being able to accommodate islets, our graphene bioscaffold will also be able to accommodate other supportive cellular therapies. Hence, we will take advantage of this by co-transplanting islets with MSCs derived from adipose tissue (AD-MSCs). AD-MSCs are able to secrete soluble factors into their surrounding microenvironment that can facilitate angiogenesis, reduce inflammation as well as modulate both the innate and adaptive immune system (24), thereby helping islets engraft, survive and function following transplantation. Indeed, studies have shown the beneficial effects of co-transplanting islets with MSCs with islets showing better structural organization, re-vascularization and reduced peri- and intra-insulitis.

In the present study, we synthesized a 3D graphene bioscaffold using template directed chemical vapor deposition (CVD) methodology and then functionalized it with a Dex coating via a polydopamine (PDA) nanolayer; the concentration of Dex was optimized to allow it to have an anti-inflammatory effect without affecting β cell function. Our graphene bioscaffold is made with interconnected macropores which are large enough to accommodate both islets and AD-MSCs evenly throughout its lattice; in turn, this will prevent cell clumping and ensure a more even distribution of islets, which will improve their survival and function (25).

### Results

### 1. Bioscaffold Synthesis and Characterization

Graphene bioscaffolds fabricated by template directed CVD methodology (FIGS. 1A-1G) retained a self-standing 3D porous structure (FIGS. 1H, 1I). By changing the dimensions of nickel foam templates, bioscaffolds with two different dimensions could be fabricated (FIGS. 1H-1O): thin graphene bioscaffolds which had a 0.5±0.1 mm thickness×50±2 mm length×25±1 mm width corresponding to a volume of 625±185 mm³ with a porosity of 80±5% and density of 0.02±0.005 mg/mm³ (FIG. 1H); and thick graphene bioscaffolds with 5±0.5 mm thickness×30±2 mm length×10±1 mm width corresponding to a volume of 1500±435 mm³ with a porosity of 80±5% and density of 0.020±0.005 mg/mm³ (FIG. 1L). SEM observation showed that graphene bioscaffolds exhibited a continuous and porous monolith structure, which copied and inherited the interconnected 3D structure of the nickel foam template (FIGS. 1I-1K, 1M-1O). Using SEM, the pore size, measured in 5 different bioscaffolds at 5 random locations, fall into 2 groups: thin graphene bioscaffold: big pores=400±50 μm and small pores=100±10 μm (FIGS. 1I-1K); thick graphene bioscaffold: big pores=800±50 μm and small pores=200±10 μm (FIGS. 1M-1O). In our work, the thin graphene bioscaffold was selected for further in vitro and in vivo studies due to its pore size distribution (100-400 μm) matching the size of islets, which typically fall between 100-400 μm (26), as well as islet:AD-MSC units. Furthermore, it can easily accommodate islets with the desired number for in vitro (20 islets in a 0.5 mm thick×1 mm length×1 mm width bioscaffold) and in vivo (250 islets in a 0.5 mm thick×5 mm length×5 mm width bioscaffold) experiments. Functionalizing bioscaffolds with a Dex coating slightly changed the bioscaffold porosity and density to 75±5% and 0.03±0.01 mg/mm³, respectively (p>0.05). Using SEM, Dex microparticles which were 6±2 μm in size, were clearly detected immobilized onto the surface of graphene bioscaffolds (FIGS. 1K, 1O). The Raman spectrum demonstrated the successful synthesis of the graphene with corresponding signals estimated from the position of the D band (1415 cm⁻¹), G band (1595 cm⁻¹) and 2D band (2800 cm⁻¹) (FIG. 1P). Using XPS, graphene bioscaffolds showed a peak corresponding to the element of carbon (C) at ˜295 eV (FIG. 10). Using a two-probe conductivity method, the conductivity of graphene bioscaffolds was also found to be 40±5 S·cm.

2. Bioscaffold Coating with Dex

Using SEM, Dex particles were found attached onto the surface of graphene bioscaffolds, and uniformly distributed, with increases in the concentration of Dex from 0.25 to 1 w/v % resulting in more Dex particles seen (FIG. 2A). Graphene-Dex bioscaffolds can release Dex for at least 14 days, with the release rate significantly increasing as the concentration of Dex increases from 0.25 to 1 w/v % (p<0.05). In the first day, the Dex release rate reached 3.96±0.21 ng/mL/h for graphene-0.25 w/v % Dex bioscaffolds; this significantly increased to 4.53±0.31 and 10.20±0.14 ng/mL/h for graphene-0.5 w/v % Dex and graphene-1 w/v % Dex bioscaffolds, respectively (p<0.05). From day 1 to 14, the Dex release rate significantly decreased to 1.04±0.85 for graphene-0.25 w/v % Dex bioscaffolds. This amount was insignificant compared to when 0.5 w/v % Dex and 1 w/v % Dex was used in bioscaffolds (day 14: 1.15±0.05 and 1.84±0.62 ng/mL/h, respectively; FIG. 2B, p<0.05).

3. In Vitro Interaction of Our Bioscaffolds with Islets±AD-MSCs

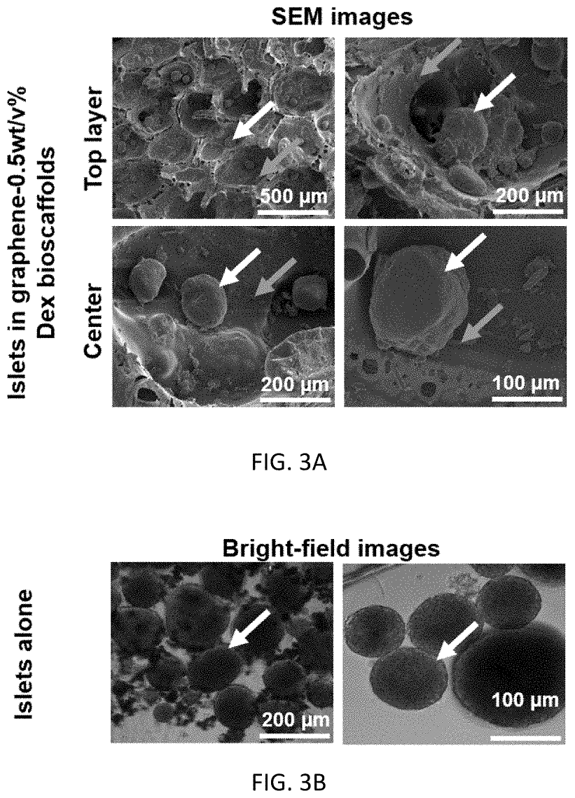

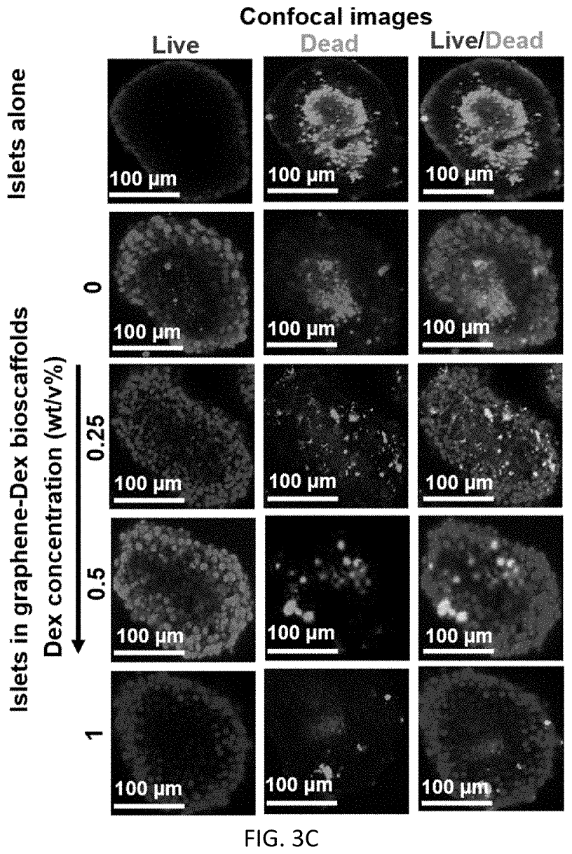

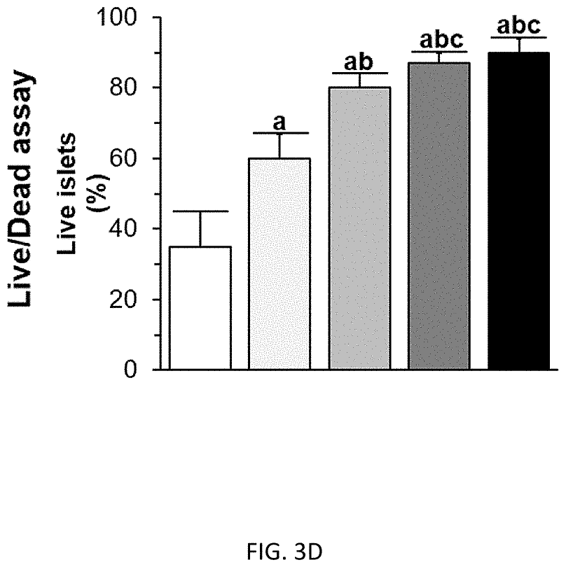

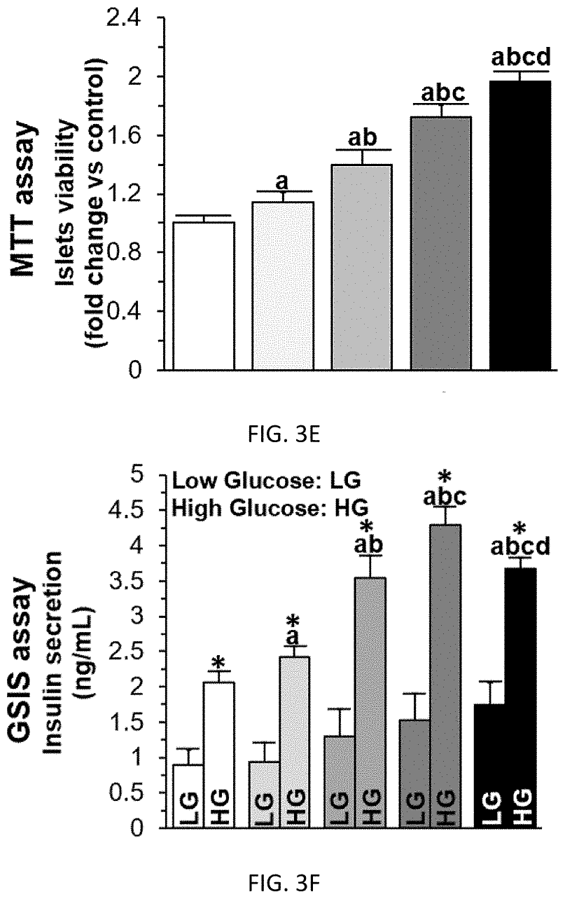

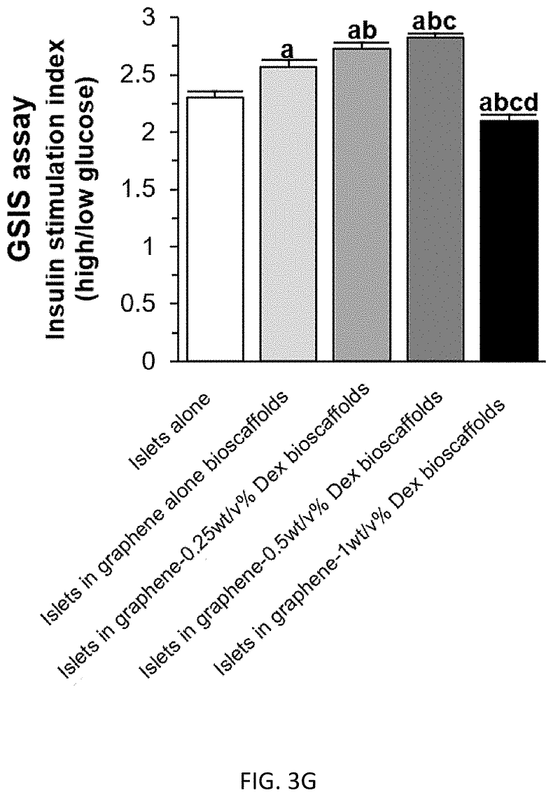

Islets seeded in graphene-0.5 w/v % Dex bioscaffolds were uniformly dispersed as shown by their presence on the top and the center of the bioscaffold. In contrast, after 7 days, islets cultured alone in the control cell culture plates showed clumping (FIGS. 3A-3C). Compared to the control, islets in graphene-Dex bioscaffolds had a significantly greater percentage of live cells (determined using the Live/Dead assay, p<0.05; FIG. 3D), viability (determined using the MTT assay, p<0.05; FIG. 3E), and functionality (determined using a GSIS assay, p<0.05; FIG. 3F). Comparing graphene-Dex bioscaffolds, cell viability was greatest for islets in graphene-1 w/v % Dex bioscaffolds, where there was an increase in live cells (90±4% vs. 35±10%, p<0.05; FIGS. 3B-3C), and cell viability (islet viability ratio: 2.0±0.2 vs. 1.0±0.3, p<0.05; FIG. 3D) compared to the control group. However, the greatest islet functionality was seen when islets were seeded into graphene-0.5 w/v % Dex bioscaffolds; here islets secreted the highest level of insulin—low glucose stimulation: 1.52±0.38 vs. 0.89±0.21 ng/mL and high glucose stimulation: 4.28±0.25 vs. 1.05±0.16 ng/mL, (p<0.05; FIG. 3F) compared to the control group. Calculation of the insulin stimulation index (SI: ratio of insulin secretion from high glucose stimulation relative to basal conditions) also showed a significant increase when islets were seeded in graphene-0.5 w/v % Dex bioscaffolds compared to the control group (2.82±0.04 vs. 2.29±0.06; p<0.05; FIG. 3G). When islet:AD-MSC units were seeded into graphene-0.5 w/v % Dex bioscaffolds, there was a significant increase in islet viability (Live/Dead assay: 94±3 vs. 87±3 and 35±10%, p<0.05; and MTT assay: 1.78±0.03 vs. 1.72±0.03 and 1±0.01 fold change vs control, p<0.05) and function (GSIS assay: 5.76±0.47 vs. 4.28±0.25 and 2.98±0.23 ng/mL insulin secretion, p<0.05; and insulin index: 5.51±0.44 vs. 2.82±0.04 and 3.23±0.25, p<0.05) compared to islets alone in graphene-0.5 w/v % Dex bioscaffolds and islets alone (i.e. control islets).

4. In Vivo Interaction of Our Bioscaffolds with Islets±AD-MSCs

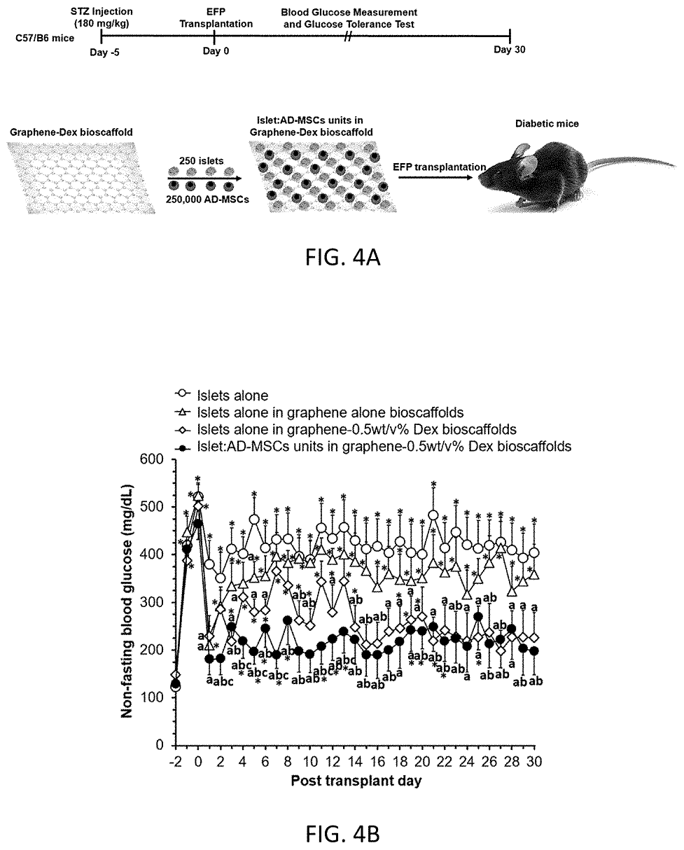

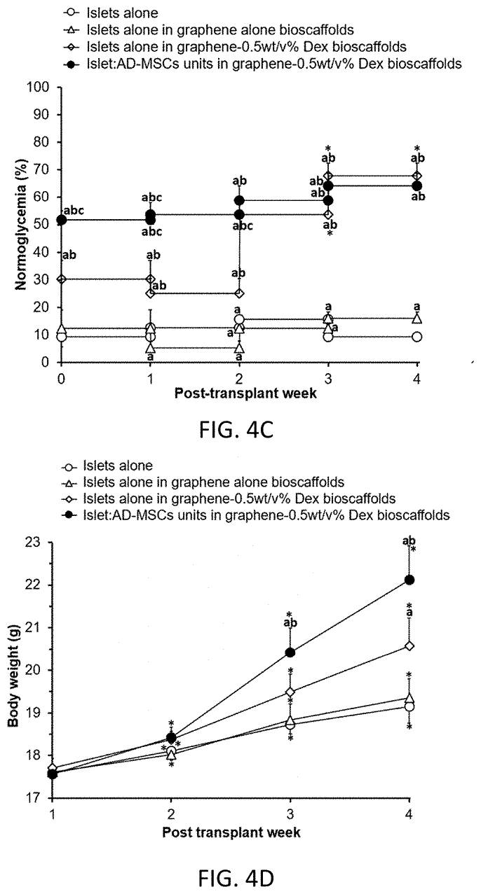

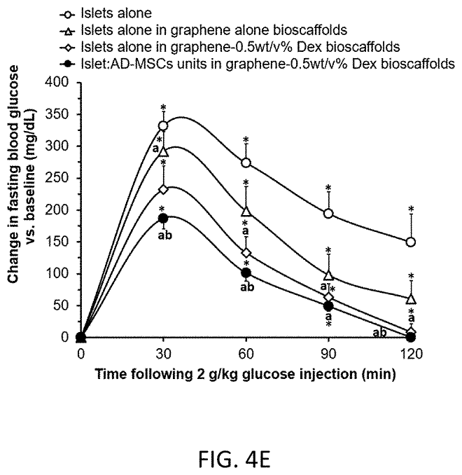

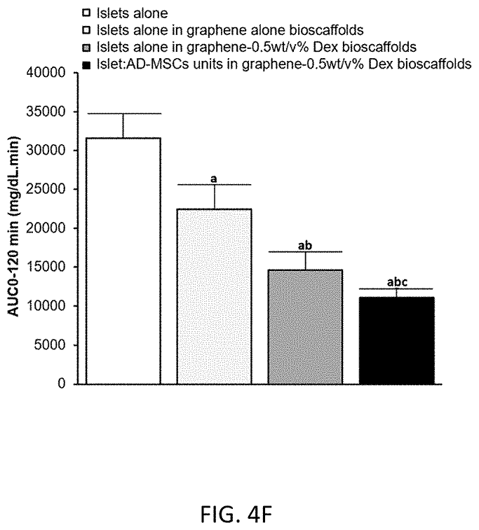

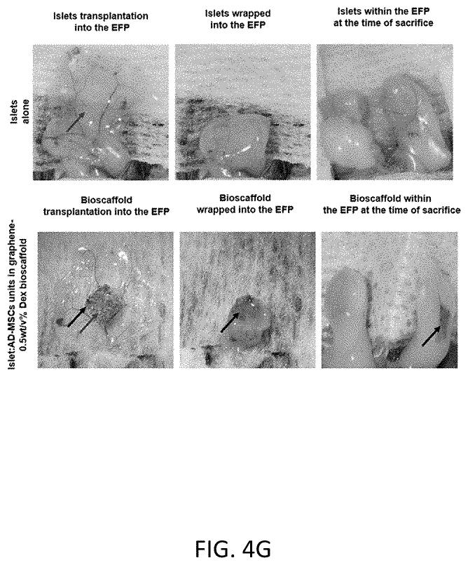

Experimental details of our in vivo experiments and transplantation procedure are outlined in FIG. 4A. Following STZ treatment, all animals became hyperglycemic (non-fasting blood glucose values increasing from 133±8 mg/dL (day −2) to 502±35 mg/dL (day 0)). Following islet transplantation, immediate re-establishment of glycemic control, with non-fasting blood glucose values returning to baseline values, was only seen in mice which received islet:AD-MSC units in graphene-0.5 w/v % Dex bioscaffolds (i.e. 181±32 vs. 380±50 mg/dL compared to control animals receiving islets alone; p<0.05; FIG. 4B), and this effect was maintained for 30 days. In contrast, diabetic mice which received islets alone or islets in graphene bioscaffolds without Dex, were not able to restore glycemic control throughout the course of this study. Interestingly, diabetic mice which received islets alone in 0.5 w/v % Dex bioscaffolds were not able to initially restore glycemic control, however, by day 14 their non-fasting blood glucose did return to baseline values and this was maintained to the end of the study at day 30. This effect was also seen when comparing the percentage of animals that exhibited normoglycemia at each week during this study (FIG. 4C). At day 0, all mice has a similar weight of 18±1 g. Following transplantation, the body weight of all mice increased; however, this increment was significantly higher for mice transplanted with islet:AD-MSC units in graphene-0.5 w/v % Dex bioscaffolds (p<0.05; FIG. 4D).

When assessing the dynamic responses to glucose challenges, mice transplanted with islet:AD-MSC units in graphene-0.5 w/v % Dex bioscaffolds demonstrated the smallest peak change in fasting blood glucose values at 30 min when compared to all other groups (p<0.05; FIGS. 4E and F). At 120-min post-glucose load, the blood glucose value returned to normal range similar to baseline (0 min), indicating normal glucose tolerance. At sacrifice, graphene-0.5 w/v % Dex bioscaffolds were tightly wrapped within the EFP and following their extraction, they were noted to be well engrafted within the fat tissue with no evidence of adhesions/fibrous bands (FIG. 4G).

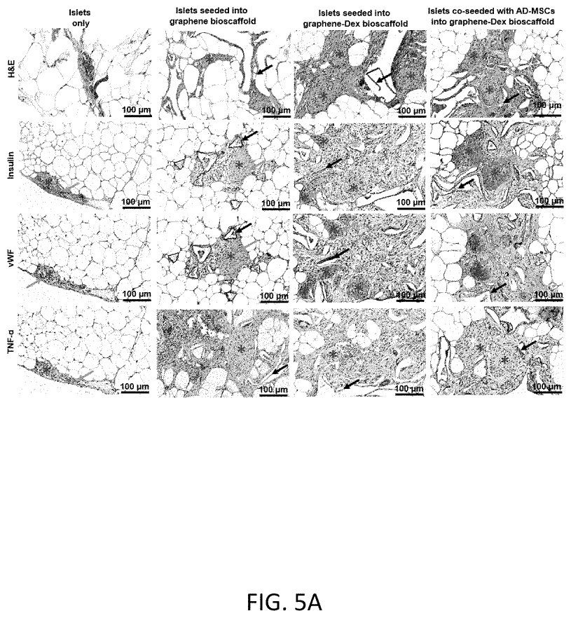

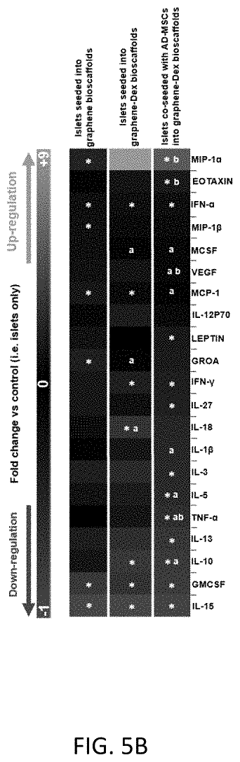

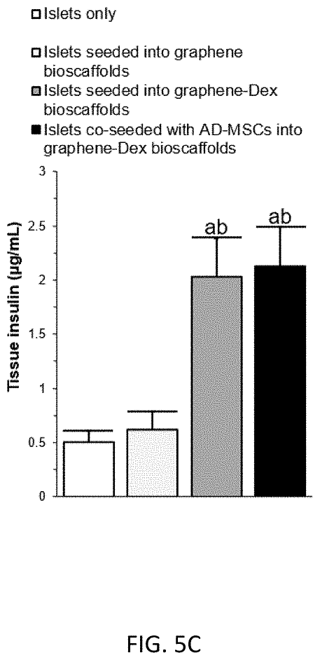

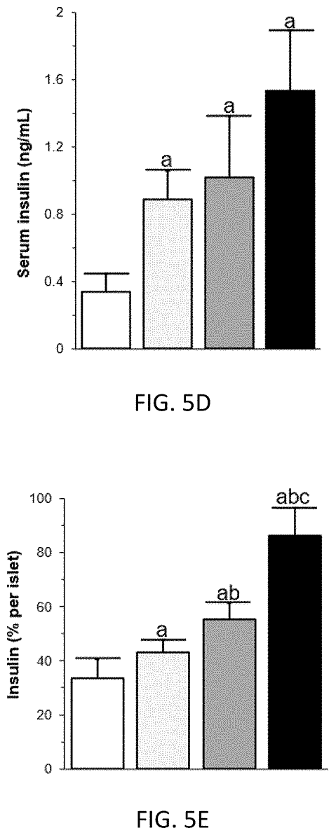

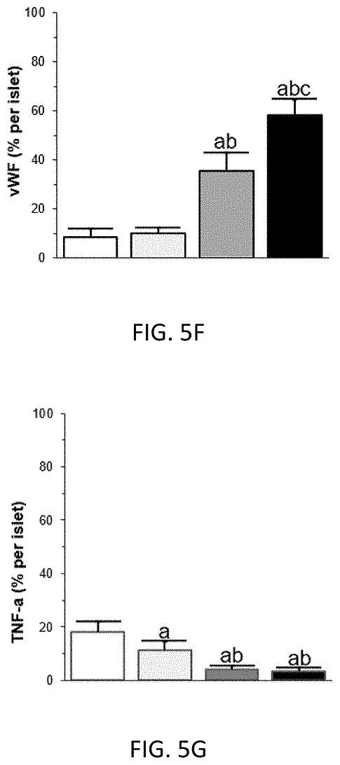

When islets were transplanted into bioscaffolds, they could be easily identified within the bioscaffolds pores (black arrows; FIG. 5A). Islet co-transplanted with AD-MSCs (i.e. islet:AD-MSC units) in graphene-0.5 w/v % Dex bioscaffolds were found on histological analysis to be significantly greater in number compared to animals which received islets alone in graphene-0.5 wt. % Dex bioscaffolds, graphene bioscaffolds alone or islets only (total islet area: 0.86±0.17 vs. 0.55±0.15 or 0.34±0.09 mm² or 0.09±0.03 mm², respectively, p<0.05; FIG. 5A). In animals which were transplanted with islets alone into the EFP, there were few intact islets visualized; furthermore, these remaining islets had lost their spherical morphology and intrinsic architecture with insulin staining cells now noted to be dispersed around the islet rather than being localized in discrete islet structures. In contrast, islets transplanted alone, or with AD-MSCs, in graphene-0.5 wt. % Dex bioscaffolds retained their native size, spherical morphology and maintained their intrinsic architecture with β cells (positive insulin staining) located in the center of the islets (FIG. 5A). When transplanted islets are healthier, they are normally intact and have a spherical structure (27). However, when islets start to die, they lose their shape due to a loss in plasma membrane integrity and cell death (28). Here, our results support the cytoprotective effect provided by AD-MSCs and graphene-Dex bioscaffolds, given that islet survival and function was improved; accordingly, islets had a more spherical and organized structure when compared to transplanted islets alone.

The greater degree of vascularity surrounding, and within, islets was further confirmed by H&E and vWF staining. Results of H&E staining showed significantly increased microvessel density for transplanted islet:AD-MSCs units in graphene-0.5 wt. % Dex bioscaffolds compared to islets transplanted alone or in graphene alone or graphene-0.5 wt. % Dex bioscaffolds (95±3 vs 11±2, 35±5, and 54±6 vessels/mm², respectively; FIG. 5A). In keeping with this, there was a significantly higher insulin staining in graphene-0.5 wt/v % Dex bioscaffolds that contained islet:AD-MSC units, compared to islets in graphene-0.5 wt/v % Dex bioscaffolds, graphene alone bioscaffolds or islets transplanted alone without a bioscaffold (percentage of insulin expression per islet: 86.5±10.2 vs. 55.3±6.2, 43.2±4.5, or 33.5±7.3%, respectively; p<0.05; FIG. 5B). Islets transplanted with AD-MSCs, in graphene-0.5 wt/v % Dex bioscaffolds demonstrated increased vascularization as evidenced by an increase in vWF staining when compared to transplanted islets alone, or islets seeded alone in graphene or graphene-0.5 wt/v % Dex bioscaffolds (percentage of vWF expression per islet: 58.5±6.2 vs. 35.7±7.2, 10±2.5, or 8.5±3.5% p<0.05; FIG. 5C). Islets transplanted alone, or with AD-MSCs, in graphene-0.5 wt/v % Dex bioscaffolds demonstrated reduced inflammation as evidenced by a reduction in TNF-α staining when compared to transplanted islets alone (percentage of TNF-α expression per islet: 3.5±1.2 or 4.3±1.1 vs. 18.2±4.1%; p<0.05; FIG. 5D). Correspondingly, the levels of insulin in the blood was significantly higher in mice that received islet:AD-MSC units in graphene-0.5 wt/v % Dex bioscaffolds compared to mice transplanted with islets alone without a bioscaffold (1.53±0.36 vs. 0.34±0.11 ng/mL; p<0.05). No significant difference was seen between islets transplanted in either graphene alone bioscaffolds or graphene-0.5 wt/v % Dex bioscaffolds (0.89±0.17 vs. 1.02±0.36 ng/mL, p>0.05; FIG. 5E). Similar results were also seen for the levels of insulin in the EFP tissue (2.13±0.45 vs. 0.50±0.01 μg/mL; p<0.05; FIG. 5F).

In addition, the EFP tissue from animals which had received islet:AD-MSC units in graphene-0.5 wt/v % Dex bioscaffolds demonstrated up-regulation of macrophage inflammatory protein-1-alpha (MIP-1α: +5.29±0.58 fold change), EOTAXIN (+1.54±0.39 fold change), interferon alpha (IFN-α: +0.89±0.01 fold change), and down-regulation of Interferon gamma (IFN-γ: −0.42±0.09 fold change), LEPTIN (−0.34±0.06 fold change), interleukin-27 (IL-27: −0.43±0.09 fold change), interleukin-3 (IL-3: −0.56±0.18 fold change), interleukin-5 (IL-5: −0.43±0.09 fold change), tumor necrosis factor alpha (TNF-α: −0.54±0.14 fold change), interleukin-13 (IL-13: −0.75±0.15 fold change), interleukin-10 (IL-10: −0.80±0.23 fold change), granulocyte-macrophage colony-stimulating factor (GMCSF: −0.82±0.29 fold change), interleukin-15 (IL-15: −0.93±0.57 fold change) when compared to control animals that received islets alone (p<0.05; FIG. 5F). Upregulated expression of MIP-1α, EOTAXIN, macrophage colony-stimulating factor (MCSF), vascular endothelial growth factor (VEGF), monocyte chemoattractant protein-1 (MCP-1), as well as down-regulation expression of interleukin-1beta (IL-1β), interleukin-5 (IL-5), TNF-α, and interleukin-10 (IL-10) was noted within the EFP tissue of animals that had received islet:AD-MSC units in graphene-0.5 wt/v % Dex bioscaffolds compared to islets seeded into graphene-0.5 w/v % Dex, or graphene alone, bioscaffolds (p<0.05; FIG. 5F).

### 5. Bioscaffold Biodegradability and Biocompatibility

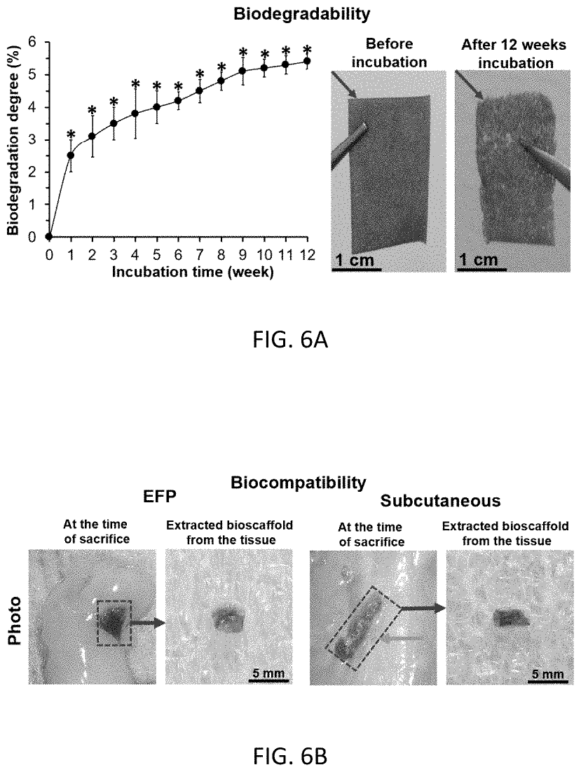

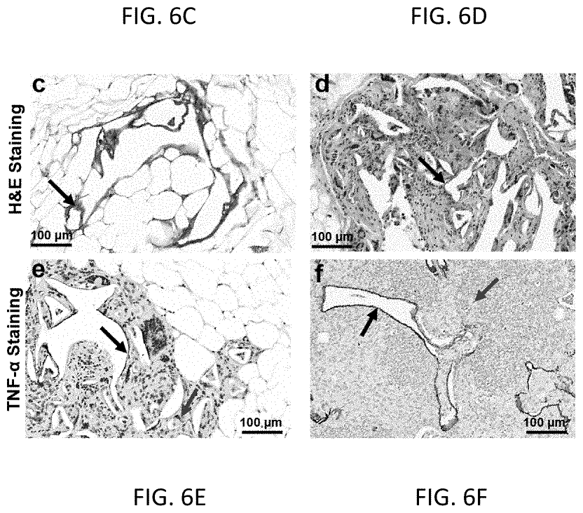

After 12 weeks incubation in PBS, the biodegradation degree of graphene-0.5 wt/v % Dex bioscaffolds was 5.40±0.23% (FIG. 6A). At 3 months post-implantation, graphene-0.5 wt/v % Dex bioscaffolds were well integrated into the EFP or subcutaneous tissue (FIG. 6B). Histological images show minimal foreign body reaction as demonstrated by the formation of fibrous tissue (FIGS. 6C-6D) and a minimal inflammatory response demonstrated by TNF-α staining, inside the bioscaffold pores and around the bioscaffolds (FIGS. 6D-6F). However, blood analysis at 3 months demonstrated no significant elevation in any parameter with average values from all animals remaining within their respective normal ranges (29, 30) (Table 1).

### DISCUSSION

Recently, graphene bioscaffolds have gained interest due to their larger interconnected macropores (i.e. super-macropores) and enhanced mechanical stability compared to traditional hydrogel and polymeric constructs (31). We therefore fabricated a graphene bioscaffold, via a template-directed CVD method that involves deposition of methane gas on a sacrificial nickel template where the porous structure of this template dictates the final architecture of the bioscaffold. When islets were seeded into these graphene bioscaffolds, the large pores (400±50 μm) enabled islets to be evenly distributed throughout its 3D matrix. In turn, this prevented islets from clumping resulting in their improved function and survival, as similarly observed in previous studies (32). In addition, islets seeded within our bioscaffolds were able to maintain their native rounded morphology, size and architecture, all of which have been shown to play a crucial role in their function and outcome following transplantation (25). Studies have also shown that the small pores (100±10 μm) of a bioscaffold, similar to the ones in our graphene bioscaffolds, allow mass transfer of metabolites and ingrowth of blood vessels resulting in an intra-islet vascular density that is comparable to native islets (9). This is supported by our in vitro (MTT, live/dead and GSIS assays) and in vivo (metabolic analysis) data showing that bioscaffolds made from graphene improved islet survival and function when compared to islets alone.

It is well established that inflammation plays a key role in decreasing islet viability and graft efficacy following their transplantation (33). Activation of the inflammatory cascade starts early, whereby pro-inflammatory mediators, such as tissue factor and high-mobility group box-1 protein, are upregulated by stressed islets following their isolation from the pancreas. Conventionally, islets are transplanted by delivery into the portal vein, which results in an innate immune attack including activation of both the coagulation and complement systems; this causes a rapid binding of activated platelets to the surface of islets and infiltration of polymorphonuclear leukocytes (PMLs) in what is commonly referred to as the instant blood-mediated inflammatory reaction (IBMIR). As a result, there is a significant loss of islets (˜60% of the islet mass) immediately following their transplantation. While implanting islets in non-endovascular locations can mitigate the IBMIR, islets ideally need a 3D matrix or scaffold to be housed in to ensure their survival; unfortunately, these can trigger a foreign body responses (FBR) (19), which again causes inflammation that can adversely affect islets. One advantage of graphene bioscaffolds is their excellent biocompatibility given that the surface of graphene sp² carbon allows for strong, non-destructive, interfacial interactions at a cellular level, which can be further improved by chemical functionalization (34). While graphene bioscaffolds did not induce a significant FBR reaction following implantation in animals (as commonly seen with synthetic bioscaffolds like PDMS), our results did show that there was a minimal inflammatory response around bioscaffolds as demonstrated on histology by cellular infiltrates and some positive TNFα staining. Unfortunately, this response was enough to likely damage the transplanted islets given that these animals remained hyperglycemic and were unable to re-establish glucose homeostasis. Hence, to address this issue of the host inflammatory response to our graphene bioscaffold, we decided to functionalize our bioscaffold with Dex since it is an immunosuppressive glucocorticoid capable suppressing inflammatory pathways (35), as well as inhibiting the expression of many inflammatory mediators (36). This approach will enable us to precisely modulate the local microenvironment following bioscaffold implantation, thereby mitigating the need for intravenous administration of Dex which is often associated with significant systemic side-effects which includes fluid retention (swelling in hands or ankles), headache, dizziness, increased blood pressure, and slow wound healing (37, 38).

In order to allow our graphene bioscaffold to be functionalized with Dex, we first applied a polydopamine coating to our bioscaffold; this served as the interface to enable our bioscaffold to then be coated with Dex given its strong adsorption properties through covalent bonding and intermolecular interactions (39, 40). Although Dex is a potent anti-inflammatory and immunosuppressive drug, at high doses it reduces insulin secretion by islets by making them insensitive to glucose (41-43). Studies have attributed this to Dex reducing the protein expression of the glucose transporter type 2 (GLUT2) transporter in beta cells (44), which then attenuates the K, glucose transport into beta cells resulting in an absent insulin response to glucose (45, 46). Hence, characterization of the release kinetics of Dex from graphene bioscaffolds is of paramount importance and in our study we found that the cumulative release profile of Dex (100-400 ng/mL over 14 days) was related to both the loading dose of the drug, as well as time, which is typical of Dex delivery platforms (47-49). In fact, although increasing the concentration of Dex on our graphene bioscaffolds was able to increase the amount of Dex released, this did not correlate with improved islet survival and function. We found that incorporation of 0.5 wt/v % Dex onto the surface of our graphene bioscaffolds resulted in the highest degree of islet survival (measured by an MTT and Live/Dead assay) and function (measured by GSIS assay) when compared to graphene bioscaffolds incorporated with higher concentrations of Dex (i.e. 1 wt/v % Dex). This was then validated in vivo in diabetic animals, with graphene-0.5 wt/v % Dex bioscaffolds loaded with islets reversing hyperglycemia and restoring glycemic control during basal conditions after 14 days, while also improving dynamic responses to glucose challenges. In addition, the EFP at day 30 from these mice contained a significantly higher amount of insulin, compared to mice transplanted with islets alone or islets in graphene alone bioscaffolds, thereby indicating that there was a higher amount of viable and functional islets within these animals. The functionalization of Dex on our graphene bioscaffolds also resulted in a greater reduction in pro-inflammatory cytokines, such as IL-1β (which is a well-established inducer of both insulin resistance and impaired pancreatic islet function (50)) and IL-18 (which induces IFN-γ (51) that then drives programmed death-ligand 1 (PDL-1) expression on islet β cells (52)), within the EFP. The lower efficiency of islet transplantation in islets alone transplanted into the EFP compared to previous studies (53-55) can be due to (i) the use of 500 islets with islet diameters in the range of 50-150 μm—this is less than 500 islet equivalents or IEQ (˜150 μm) used in previous studies (53-55) and (ii) higher initial blood glucose of our animals at the transplantation day (i.e. 502±35 mg/dL) compared to previous studies (i.e. 250-500 mg/dL) (53-55)—this would mean that our animals had a higher degree of dysglycemia and were more dependent on the success of their islet transplant. While graphene-0.5 wt/v % Dex bioscaffolds were eventually able to restore hyperglycemia, they did so only after 14 days, which resulted in diabetic animals for the first 2 weeks following transplantation not being able to regain glucose homeostasis. Hence, to improve islet engraftment during this time, we added a supportive cellular therapy in the form of AD-MSCs; these cells have been shown to enhance the survival and function of transplanted islets by promoting their revascularization as well as suppressing any localized inflammatory responses. In order for AD-MSCs to be effective, they need to be in close proximity to islets to first sample the surrounding microenvironment and then modulate it to facilitate islet engraftment by releasing the appropriate paracrine factors into the same local microenvironment. Hence, to ensure that AD-MSCs were “spatially located” next to islets at the time of transplantation, we first co-culture them together to create islet:AD-MSC units. These islet:AD-MSC units were then seeded in graphene-0.5 wt/v % Dex bioscaffolds which resulted in animals having an almost immediate response with glycemic control being re-established with 24 h and then sustained for 30 days. Taken together, these results would suggest that graphene-0.5 wt/v % Dex bioscaffolds are essential for islet survival and that the initial engraftment and function of islets can be improved when they are seeded into these bioscaffolds as islet:AD-MSC units compared to when they seeded as islets alone. Significant differences between islets and islet:AD-MSC units were noted in animals when assessing glycemic control and the percentage of animals that became normoglycemic over the first 14 days as well as in the long term as shown by animal body weight over 30 days. While there was also the suggestion that islets within islet:AD-MSC units demonstrated better dynamic responses to glucose challenges, healthier morphology and higher levels of serum insulin, these changes were not significant. One explanation for this was that the IPGTT test was done at 14 days and the molecular assessment of the transplants were done at 30 days (i.e. the time of animal sacrifice and tissue harvesting) and by this time the islets in both groups were likely fully established and engrafted (as demonstrated in their basal glycemic control) and that any differences may have been more significant at earlier time points. Transplanted islets also demonstrated reduced inflammation shown by a reduced TNF-α expression on histology as well as down-regulation of pro-inflammatory cytokines IL-1β (56), IL-10 (57), IL-5 (58), and TNF-α (59) in the tissue lysate of the islet transplant. Together, these results show the ability of this approach to help islet engraftment at the site of transplantation by lowering the islet inflammation.

The current clinical approach for islet transplantation is to infuse islets into the liver, however, this renders them irretrievable (60) as well as exposing them to adverse conditions such as hypoxia and inflammation. Given these challenges associated with the liver, other extravascular sites are now being evaluated such as the omentum or subcutaneous space; however, for islets to be transplanted and survive in these areas, they need a supportive structure such as a bioscaffold. While the subcutaneous space can be easily accessed for bioscaffold implantation, the omentum also has several promising attributes which includes its large well vascularized surface area (61). Both sites also have the ability to accommodate 3D bioscaffolds (11, 39, 40), as well as the ability to retrieve these platforms should something adverse happen. Furthermore, human clinical trials are already underway examining the feasibility of the omentum as a site for polydimethylsiloxane (PDMS) based bioscaffolds for islet transplantation (61). In the present study, we therefore examined the biocompatibility of our graphene-Dex in both the epididymal fat pad (EFP) (which in mice is used as a surrogate site for the omentum (13, 61, 62)) and the subcutaneous space. At both sites, our graphene bioscaffold showed that they were well integrated into the surrounding tissues with minimal, if any, evidence of ongoing/active inflammation or fibrosis; this is essential to ensure the long-term safety and integrity of our implants (63). In addition, all blood biochemistry and electrolyte markers were within their normal range with no perturbations noted.

Given that our data shows that our graphene-Dex bioscaffold can augment islet survival and function with excellent biocompatibility, future research will aim to examine using this platform at both these sites with human islets in appropriate animal models over the short and long term. In humans, islets are typically given at 5,000 islets per kg (i.e. a weight based approach) which, on average, translates to approximately 350,000-450,000 islets (64). The thin graphene bioscaffolds (0.5 mm thick×5 mm length×5 mm width) which were used in our experiments can accommodate up to 500 islets. However, using our established CVD protocol we can easily manufacture bigger graphene bioscaffolds thereby easily ensuring the clinical use of graphene bioscaffolds with a volume >100 cm³, which we have calculated can accommodate the required number of islets for human transplantation. Given that islets will also require protection against inflammatory reactions triggered by the foreign body response to the bioscaffold itself, combining our graphene bioscaffolds with Dex can address this issue to ensure islets have the optimal microenvironment following their transplantation. While graphene has recently been used for drug delivery systems, and ultrasensitive electrochemical biosensors for detection of biomolecules, this is the first study to show its potential use as a bioscaffold for a cellular therapy (i.e. pancreatic islet transplantation). Hence, graphene is well placed to be translated into human clinical trials following further optimization and validation.

The in vitro biodegradability rate was low such that bioscaffolds would be expected to provide a stable space to accommodate islets until engraftment. However, if bioscaffolds induce a strong inflammatory response, this will result in rapid biodegradation and loss of mechanical and structural integrity. Hence, to alleviate this effect, we can also enhance the biodegradability of our bioscaffolds by reducing the methane gas concentration in the CVD reactor during graphene synthesis or using a shorter growth time. Nevertheless, the in vitro biodegradability results need to be interpreted with caution as they do not completely predict the in vivo biodegradability (65, 66). The in vivo biodegradation rate will be strongly dependent on the surrounding blood flow, bioscaffold implantation site, oxygen supply, pH values as well as the amount of water and ion content in the surrounding microenvironment (67). Hence, although we expect >5 years stability of our bioscaffolds, a long-term in vivo biodegradability study will need to be performed to fully assess this.

In this study, syngeneic islet transplantation has been studied in which mice from the same strain have been used as both the donor and recipient. This model allows us to evaluate the innate immune response towards the bioscaffold, and more specifically the foreign body response towards the biomaterial from which our bioscaffold is constructed. This approach is also very pertinent to the autologous islet transplant setting. In contrast, for allogenic islet transplants, we will need to use a different model in which we have an MHC mis-match, thereby allowing us to evaluate and focus more on the adaptive immune response. Future studies will therefore evaluate this setting as well as the ability of our bioscaffold to support human islet cargo in nude or immunocompromised mice models. Future work will also examine further optimization of our graphene-0.5 wt/v % Dex bioscaffold, with the incorporation of extracellular matrix (ECM) molecules or growth factors, in animal models which can accommodate human islets (i.e. NSG mice). We will also focus on the long-term outcome (e.g. >6 months), from both the biological (i.e. maintenance of basal and dynamic glycemic control) and biocompatibility perspective, using our graphene-0.5 wt/v % Dex bioscaffold for islet transplantation. This bioscaffold can be produced in accordance with GMP guidelines which will also facilitate its clinical translatability. Although not performed in the current study, mechanical tests can be performed in future to determine the tensile strength and percentage of elongation of both thin and thick bioscaffolds. However, our graphene bioscaffolds (both thin and thick) demonstrated adequate flexibility for in vitro culture experiments and in vivo implantation studies. If any wear or fracture of our bioscaffolds are encountered in vivo, graphene-polymer composite bioscaffolds can also be developed in future; however, the changes in their biological characteristics will need to be studied.

In summary, this combination approach of using a graphene bioscaffold that can be functionalized for localized Dex delivery for 3 weeks, together with AD-MSCs therapy, can significantly improve the survival and function of transplanted islets resulting in a rapid and stable restoration glycemic control in diabetic animals following their transplantation.

TABLE 1
Blood metabolic, chemistry, and liver panels from mice
that had been implanted with graphene-0.5 wt/v %
Dex bioscaffolds in EFPs for 3 months. The normal
range for each parameter is also listed in the table.

| | | |
|---|---|---|
| | Mice implanted | |
| | with graphene- | |
| | 0.5 wt/v % Dex | |
| Test | bioscaffolds | Normal range |

Metabolic panel

| | | | |
|---|---|---|---|
| Sodium | 151 ± 1 | 146-151 | mmol/L |
| Chloride | 109 ± 2 | 107-111 | mmol/L |
| Carbon Dioxide | 18 ± 2 | 20-29 | mmol/L |
| Potassium | 4.1 ± 0.2 | 3.0-9.6 | mmoL/L |
| Blood Urea Nitrogen | 24 ± 1 | 20.3-24.7 | mg/dL |
| Creatinine | 0.18 ± 0.02 | 0.1-1.1 | mg/dL |

Chemistry panel

| | | | |
|---|---|---|---|
| Calcium | 9.7 ± 0.1 | 8.9-9.7 | mg/dL |
| Phosphorous | 8.3 ± 0.4 | 4.2-8.5 | mg/dL |
| T.Protein | 5.2 ± 0.1 | 4.5-6.5 | g/dL |
| Albumin | 3 ± 0.1 | 2.5-2.8 | g/dL |

Liver panel

| | | | |
|---|---|---|---|
| Alkaline phosphatase | 65 ± 1 | 44-147 | IU/L |
| Aspartate Aminotransferase (AST) | 34 ± 5 | 10-45 | U/L |
| Alanine Aminotransferase (ALT) | 26 ± 2 | 10-35 | U/L |
| Total Bilirubin | 0.3 ± 0.1 | 0-1.0 | mg/dL |

### Example 2

### Materials and Methods

### 1. Bioscaffold Fabrication and Characterization

Graphene bioscaffolds were fabricated using template-directed chemical vapor deposition (CVD) as previously described (68). Briefly, a nickel foam (Goodfellow Inc., USA) with 0.45 g·cm⁻³ density, 95% porosity, 40 mm length, 30 mm width and 1.6 mm thickness (thin) or 6 mm thickness (thick), was used as the template for the CVD growth of graphene. The thin or thick nickel foam was placed in a CVD furnace (Aixtron Black Magic, USA) dedicated to graphene synthesis with methane (CH₄) and hydrogen (H₂) available as the process gases. The foam was first annealed at 1000° C. for 5 min under argon (Ar) and H₂ atmosphere with the flow rates of 500 and 200 s.c.c.m., respectively to clean its surfaces and eliminate the surface oxide layer. The CH₄ gas, with the flow rate of 50 s.c.c.m, was then introduced into the reaction chamber. After 5 min of reaction-gas mixture flow, the foam was rapidly cooled down to room temperature under Ar and H₂ atmosphere with flow rates of 500 and 200 s.c.c.m., respectively. Following graphene growth, the nickel-graphene foams were coated with a thin polymethyl methacrylate (PMMA) layer to support the graphene structure and prevent its structural failure when the nickel foams are etched away in next step. The nickel-graphene foams were therefore dip-coated with polymethyl methacrylate (PMMA, average molecular weight of 996,000, Thomas Scientific, USA) dissolved in ethyl L-lactate (4 wt. %; Alfa Aesar, USA), and then baked at 180° C. for 2 h to obtain a nickel-graphene-PMMA foam. This process solidifies PMMA to form a thin film on the graphene surface. Next, the nickel-graphene-PMMA foam was immersed into a mixture of Iron(III) chloride (FeCl₃; Fischer Scientific, USA) and hydrochloric acid (HCl; Fischer Scientific, USA) with the ratio of FeCl₃/HCl:1M/1M solution at 80° C. for 72 h to dissolve the nickel. Finally, graphene bioscaffolds were obtained by dissolving the PMMA with acetone (FIGS. 1A-1G). The graphene bioscaffolds were then washed 3 times with distilled water to ensure removal of all before being sectioned to obtain cubes measuring 0.5 mm thick×5 mm length×5 mm width (thin) or 3 mm thick×5 mm length×5 mm width (thick). The thin graphene bioscaffold was used in our in vitro and in vivo experiments. Next, microstructural, chemical, and physical characteristics of bioscaffolds were analyzed (see Supporting Information).

### 2. Bioscaffold Coating

Dex (Enzo life sciences, USA) was incorporated onto the surface of graphene bioscaffolds using a polydopamine coating as we previously reported (39, 40). Polydopamine was used as an adhesive (69) and biocompatible (70) coating nanolayer to hold Dex onto bioscaffolds. In brief, bioscaffolds were immersed in a dopamine solution (2 mg/ml in 10 mM Tris, pH 8.5 in the dark). Dex was subsequently added to the dopamine solution at the desired percentage (0.25, 0.5, and 1 w/v %, which results in a total mass of 45, 90, and 180 mg Dex per bioscaffold (0.5 mm thick×5 mm length×5 mm width), respectively). The mixture containing the graphene bioscaffold, dopamine solution and Dex was then placed on a tube rotisserie at 18 rpm for 30 min at 25° C. to self-polymerize the dopamine. The graphene-Dex bioscaffolds was washed 3 times in phosphate buffered saline (PBS; Gibco, USA) to ensure removal of all chemicals. A Kimwipe was then used to wick away any residual water before leaving bioscaffolds to dry at room temperature for further analysis. To avoid any reaction of polydopamine with PBS, and Dex release, during the washing process we minimized the process of washing graphene-Dex bioscaffolds with PBS (<15 s). Here, the graphene bioscaffolds coated with 0.25, 0.5, and 1 wt/v % Dex were called graphene-0.25 wt/v % Dex, graphene-0.5 wt/v % Dex, and graphene-1 wt/v % Dex bioscaffolds, respectively. To evaluate the release kinetic of Dex, bioscaffolds were incubated in benzalkonium chloride (BKC, 5 mL, 1 wt/v %, Sigma-Aldrich, USA) for 14 days, with samples collected at day 1, 2, 3, 7, 10 and 14 and the Dex concentration at each of these time points was measured using a Dex ELISA kit (Neogen, USA). BKC was used as a solubilizing agent to promote a high sink condition and mimic the clearance of the steroid in vivo as previously reported for the measurement of Dex release from different materials (55). Dex-free graphene bioscaffolds were used as the control group.

3. In Vitro Interaction of Our Bioscaffolds with Islets±AD-MSCs

All assays were carried out on islets alone or islet:AD-MSC units, i.e. islets co-cultured with AD-MSCs for 24 h in a 1:100 ratio according to our previously published protocol (71), manually picked under a microscope, and seeded directly into bioscaffolds (20 islets or 20 islet:AD-MSC units) or into a 96-well plate. For all in vitro experiments, isolated islets were individually counted and manually picked-up under a microscope to achieve a density of 20 islets/well (20 islets in 200 μL of complete medium: RPMI-1640 medium (Gibco, USA) supplemented with 10% fetal bovine serum (FBS; Invitrogen, USA) and 50 U/mL penicillin-50 μg/mL streptomycin (P/S; Invitrogen, USA) were added into 96-well non-adherent tissue culture plates. All experiments were performed in n=5 on the following experimental groups: 1) islets alone or islets in 2) graphene alone bioscaffolds, 3) graphene-0.25 wt/v % Dex bioscaffolds, 4) graphene-0.5 wt/v % Dex bioscaffolds, 5) graphene-1 wt/v % Dex bioscaffolds, and 6) islet:AD-MSC units in graphene-0.5 wt/v % Dex bioscaffolds (i.e. our optimized bioscaffold configuration).

3.1. Islet Isolation: Pancreatic islets were isolated from the pancreas of C57/B6 mice (female, 6-8 week-old, Charles River Laboratories, USA), as we previously described (28). Briefly, mice were anesthetized and then euthanized by cervical dislocation. The abdomen was opened, the bowel was moved to the left side and the pancreas and common duct were then exposed. A hemostat clamp was placed on either side of the small intestine and the pancreas was inflated through the bile duct with a 30-gauge needle and a 5 mL syringe containing 3 mL of cold collagenase solution. The pancreas was then removed from the body and placed in a vial containing 2 mL of collagenase. Digestion lasted for 10 min and then the pellet was re-suspended in Ficoll of different densities. The islet layer was identified, picked and washed with Hank's balanced salt solution (HBSS; Gibco, USA) supplemented with 0.1% bovine serum albumin (BSA; Gibco, USA). Islets were then individually counted and picked manually under a microscope.

3.2. AD-MSCs Isolation: AD-MSCs were isolated from the adipose tissue of C57BL6 mice (female, 6-8 week-old, Charles River Laboratories, USA), as described in our previous works (39, 40). Briefly, mouse adipose tissue was obtained from the lower abdomen of mouse. Harvested adipose tissue was then washed with sterile PBS, minced with scissors and then digested with 1 mg/mL type I collagenase (Sigma-Aldrich, USA) in serum-free medium at 37° C. for 3 h. The digestion was then inactivated with an equal volume of complete medium. All samples were then filtered through a 100 μm mesh filter to remove any debris. The cellular pellets were collected and then re-suspended in complete medium in a humidified incubator at 37° C. with 5% carbon dioxide. AD-MSCs from passage number 3-5 were used in our studies.

3.3. Creating Islet:AD-MSC units and Bioscaffold Seeding of Cellular Therapies: Islets were cultured in a complete medium containing: Low glucose Dulbecco's Modified Eagle's medium (DMEM; glucose concentration: 1 g/L, Gibco, USA) supplemented with 10% fetal bovine serum (FBS; Invitrogen, USA) and 50 U/mL penicillin-50 ug/mL streptomycin in a humidified incubator under normal conditions (0.2 mM (20%) 02 and 5% CO₂) at 37° C. The cultured AD-MSC were enzymatically detached from culture plates upon reaching 70-80% confluence using 0.25% trypsin solution in order to obtain a single cell suspension and re-suspended in complete medium. AD-MSCs were then counted on a hemocytometer under a bright-field microscope and co-cultured with AD-MSCs for 24 h in a 1:100 ratio according to our previously published protocol (71). Bioscaffolds were sterilized by soaking them in 70% ethanol for 0.5 h after which time they were rinsed 3 times in sterilized PBS. Islets alone or islet:AD-MSC units (i.e. islets co-cultured with AD-MSCs for 24 h in a 1:100 ratio) were then hand-picked and seeded into sterilized bioscaffolds, achieving a density of 20 islets in 200 μL complete medium per bioscaffold (cubes measuring 0.5 mm thick×1 mm length×1 mm width); these were then placed within each well of a 96-well plate.

3.4. Islet Viability: Following procedures previously described in (40), we determined the viability of islets using a 3-(4,5-Dimethylthiazol-2-yl)-2,5-diphenyltetrazolium bromide (MTT) assay. Here, 50 μL of MTT solution (0.5 mg/mL) was added to the complete medium in each well and left to incubate at 37° C. for 4 h. Water-soluble MTT is taken up by viable cells and converted to an insoluble formazan. Next, 200 μL of DMSO (to dissolve the formazan) was added to each well and left at 37° C. for a further 10 min before the absorbance was measured at 570 nm using a microplate spectrophotometer system—the absorbance directly relates to the number of viable cells present (72). Cell viability was determined using the Eq. 4:

$$Cell viability = \frac{{OD}_{sample}}{{OD}_{control}} \qquad ( Eq . 4 )$$

ODₛₐₘₚₗₑ is the optical density (absorbance) of islets and OD_{controi} is the optical density (absorbance) of islets that were not exposed to any bioscaffolds. To mitigate potential sources of errors caused by the effect of graphene or Dex on MTT readings, graphene alone and graphene-Dex bioscaffolds without any MTT solution were used as background controls.

3.5. Islet Function: Islets seeded in graphene-Dex bioscaffolds were cultured in a humidified incubator under normal conditions (37° C./5% CO₂/20% O₂). After 7 days of incubation, the ability of islets to secrete insulin was assessed by exposing them to low glucose (i.e. basal conditions) and high glucose (i.e. stimulated conditions) media (200 μL/well). Islets were incubated in Krebs-Ringer Buffer (KRB; Sigma-Aldrich, USA) spiked with 2.8 mM glucose (low) for 2 h followed by 16.7 mM glucose (high) for 2 h at 37° C./5% CO₂/20% O₂ (73). The supernatants were collected at the end of incubation for both basal and stimulated condition and insulin levels were quantified using a mouse insulin ELISA kit (Mercodia Developing Diagnostics, USA). The total insulin content of islets (i.e. 20 islets) was then normalized to present the amount of insulin secreted per islet.

3.6. Islet Imaging: Following procedures previously described in (40), we labelled islets using Hoechst 33342 (for live cells, ThermoFisher Scientific, USA), propidium iodide (PI; for dead cells, ThermoFisher Scientific, USA), and fluorescein diacetate (FDA; for AD-MSCs, ThermoFisher Scientific, USA). The culture medium was removed under a bright-field microscope, the staining solution (Hoechst 33342 (504/well), PI (50 μL/well) and FDA (50 μL/well)) was added and then left to incubate in darkness at 5% CO₂ and 37° C. for 20 min. At the end of the incubation time, the staining solution was removed and cells were washed three times with PBS. The live cell imaging solution (ThermoFisher Scientific, USA) was then added to each well before imaging. Images were acquired with a Zeiss LSM710 Confocal Microscope at a magnification of 20× and figures were created with the FIJI software (ImageJ, GNU General Public License). Islets were further visualized with SEM by acquiring images from 3-5 random locations within each bioscaffold. For SEM imaging, sectioned bioscaffolds were washed 3 times with PBS, fixed using 4% paraformaldehyde for 0.5 h at room temperature and then dehydrated in graded ethanol solutions (50, 70, 90 and finally 100% absolute ethanol). All bioscaffolds were then dried at room temperature, sectioned, sputter coated with Au—Pd and then analyzed with SEM.

4. In Vivo Interaction of Our Bioscaffolds with Islets±AD-MSCs

4.1. Diabetes induction and islet transplantation: All procedures were performed in accordance with the regulations approved by the Institutional Animal Care and Use Committee (IACUC) of Stanford University. The study was conducted on a diabetic mouse model (C57BL/6, Male, 6-8 weeks, Charles River Laboratories, USA). To induce diabetes, each mouse received an intra-peritoneal injection of streptozotocin (STZ) at the dose of 180 mg/kg; this technique is a well-established model for inducing diabetes in rodents and hence for studying islet transplantation (24, 71, 74-77) given that STZ selectively causes destruction of insulin producing β cells within pancreatic islets (78). Tail vein blood was collected to measure blood glucose concentrations with a portable glucose meter (Bayer Contour Glucose Meter, USA) for the next 5 days. Animals were determined as diabetic and ready for islet transplantation when they became hyperglycemic (non-fasting blood glucose >350 mg/dL on 2 consecutive days as previously documented (79)). These diabetic recipient mice were then prepared for surgery and anesthetized with 2% isoflurane in oxygen. The abdomen was shaved and swabbed with betadine and isopropanol to sterilize the skin. Next, a 7 mm incision was made through the peritoneal wall in the midline close to the genital area. The EFP on one side was gently grabbed and pulled out from the abdominal cavity using forceps. A bioscaffold piece (cubes measuring 0.5 mm thick×2.5 mm length×2.5 mm width) was placed on the unfolded EFP (all bioscaffolds were sterilized by incubating them in 70% ethanol for 30 min and washed three times under sterile conditions in PBS before they were transplanted). A total of 250 islets alone or islet:AD-MSC units mixed with 5 μL Matrigel (Fischer Scientific, USA) were then seeded into each bioscaffold. The EFP was folded and sutured around the bioscaffold before being placed back into the abdominal cavity of the animal. This procedure was repeated for the contralateral EFP, thereby achieving 500 islets alone or islet:AD-MSC units seeded into 2 bioscaffolds for each recipient animal. Control animals (i.e. islets only) also received 500 islets using a similar technique, with 250 islets alone seeded into the EFP on each side of the animal; this amount has been previously shown to be sub-therapeutic. At the end of the surgery, the abdomen was closed and animals were left to recover for 24 h. A total of 4 experimental groups were used (n=8 animals per group): Group 1: Mice transplanted with islets alone; Group 2: Mice transplanted with islets in graphene alone bioscaffolds; Group 3: Mice transplanted with islets alone in graphene-0.5 wt/v % Dex bioscaffolds; Group 4: Mice transplanted with islet:AD-MSC units in graphene-0.5 wt/v % Dex bioscaffolds.

4.2. Blood Glucose Measurement: Blood glucose was monitored daily from the tail vein blood for 30 days post transplantation. Mice were considered normoglycemic when blood glucose levels were less than 200 mg/dL (80). Time to re-establish normoglycemia was defined as the number of days required to re-establish blood glucose levels consistently lower than 200 mg/dL.

4.3. Intraperitoneal Glucose Tolerance Tests (IPGTT): The function of the islets were examined further with IPGTT performed at day 14 post transplantation. Mice were fasted overnight before receiving an intraperitoneal glucose bolus (2 g/kg). Blood glucose levels were monitored at 0, 30, 60, 90, and 120 min after injection. The changes in blood glucose levels at 30, 60, 90, and 120 min time points versus baseline (0 min time point) were presented. The slope of blood glucose changes versus time after injection and the area under the curve (AUC) were calculated between transplant groups.

4.4. Histological Analysis: Sections were prepared for histological and immunohistochemical analyses to determine islet structure and viability (Hemotoxylin and Eosin (H&E) and insulin staining), evidence of vascularization (von Willebrand Factor (vWF) antibody staining) and evidence of inflammation (tumor necrosis factor alpha (TNF-α) staining) via standard procedures. The stained sections were then imaged using a NanoZoomer slide scanner 2.0-RS (Hamamatsu, Japan). Results were analyzed using FIJI Image J software from 5 different sections through the EFP of each animal.

4.5. Molecular Analysis: At day 30 post-transplantation, mice were euthanized and serum and tissue (i.e. the EFP with or without graphene-Dex) samples collected to determine insulin levels (insulin ELISA kit; Mercodia). The frozen EFP tissue was then homogenized as follow: tissue samples were placed in a homogenization buffer at a ratio of 1 kidney/1 mL buffer; the buffer contained a protease inhibitor combination (Sigma Aldrich, USA) including 4-(2-Aminoethyl)benzenesulfonyl fluoride hydrochloride (AEBSF, 2 mM), Aprotinin (0.3 μM), Bestatin (116 μM), trans-Epoxysuccinyl-L-leucylamido(4-guanidino)butane (E-64, 14 μM), Leupeptin (1 μM) and ethylenediaminetetraacetic acid (EDTA, 1 mM) in tissue protein extraction reagent (ThermoFisher Scientific, USA) containing phenylmethylsulfonyl fluoride (PMSF). All homogenized EFP samples were sonicated 3 times for a total of 8 s (Branson SLPe) mixed for 45 min at 4° C., before then being centrifuged at 15000 rpm (for 15 min at 4° C.). The tissue supernatant was then collected and the insulin content measured (mouse insulin ELISA kit; Mercodia); results were normalized per EFP for each mouse. The level of tissue cytokines was also measured using a mouse multiplex ELISA (eBiosciences/Affymetrix/Fisher). In brief, beads were first added to a 96 well plate and washed (Biotek ELx405). Samples were then added to the plate containing the mixed antibody-linked beads and incubated at room temperature for 1 h followed by overnight incubation at 4° C. on a plate shaker (500 rpm). Biotinylated detection antibody was then added, after which the plates were incubated at room temperature for 75 min on the plate shaker (500 rpm). Next, the samples were washed and streptavidin-PE added followed by incubation of the plate 30 min at room temperature on the plate shaker (500 rpm). The plate was then washed and a reading buffer added to all the wells. Finally, a Luminex Flex 3D instrument was used to read the plates with a lower bound of 50 beads per sample per cytokine. Control assay beads (Radix Biosolutions) were added to all wells.

### 5. Bioscaffold Biodegradability and Biocompatibilitv

5.1. Biodegradability: Graphene-0.5 wt/v % Dex bioscaffolds (our optimized bioscaffold) were weighed (dry weight: W_{d1}) and then incubated in PBS at 37° C. for 3 months. Every week, bioscaffolds were removed from PBS, dried overnight and re-weighed (dry weight (W_{d2})). The degree of bioscaffold biodegradation was calculated as follows: ((W_{d1}−W_{d2})/W_{d1}×100) (11).

5.2. Biocompatibility: Graphene-0.5 wt/v % Dex bioscaffolds were implanted into the EFP and subcutaneous tissue of C57/B6 mice. After 3 months, mice were sacrificed and the EFP and subcutaneous tissue containing the implanted bioscaffolds were harvested for macroscopic and microscopic (i.e. histology with H&E and IHC with TNF-α) examination, specifically looking at the bioscaffold and surrounding tissue. Blood samples were also collected for routine analysis (i.e. electrolyte, metabolic, chemistry, and liver panels) (11).

### 6. Statistical Analysis

All experiments were performed in n=5 for in vitro or n=8 for in vivo experiments, and all results were expressed as mean±standard error of the mean. Statistical analysis of all quantitative data was performed using a one or two-way ANOVA (Analysis of Variance) with post-hoc Tukey test (Astatsa.com; Online Web Statistical Calculators, USA) with any differences considered statistically significant when p<0.05.

### REFERENCES

1. S. Pellegrini, E. Cantarelli, V. Sordi, R. Nano, L. Piemonti, The state of the art of islet transplantation and cell therapy in type 1 diabetes. Acta diabetologica 53, 683-691 (2016).
2. L. Moberg, The role of the innate immunity in islet transplantation. Upsala journal of medical sciences 110, 17-56 (2005).
3. A. J. Shapiro, C. Ricordi, B. Hering, Edmonton's islet success has indeed been replicated elsewhere. The Lancet 362, 1242 (2003).
4. M. M. Coronel, C. L. Stabler, Engineering a local microenvironment for pancreatic islet replacement. Current opinion in biotechnology 24, 900-908 (2013).
5. A. Rajab, Islet transplantation: alternative sites. Current diabetes reports 10, 332-337 (2010).
6. T. Kheradmand, S. Wang, R. F. Gibly, X. Zhang, S. Holland, J. Tasch, J. G. Graham, D. B. Kaufman, S. D. Miller, L. D. Shea, Permanent protection of PLG scaffold transplanted allogeneic islet grafts in diabetic mice treated with ECDI-fixed donor splenocyte infusions. Biomaterials 32, 4517-4524 (2011).
7. E. Pedraza, A.-C. Brady, C. A. Fraker, R. D. Molano, S. Sukert, D. M. Berman, N. S. Kenyon, A. Pileggi, C. Ricordi, C. L. Stabler, Macroporous three-dimensional PDMS scaffolds for extrahepatic islet transplantation. Cell transplantation 22, 1123-1135 (2013).
8. A. M. Smink, D. T. Hertsig, L. Schwab, A. A. van Apeldoorn, E. de Koning, M. M. Faas, B. J. de Haan, P. de Vos, A retrievable, efficacious polymeric scaffold for subcutaneous transplantation of rat pancreatic islets. Annals of surgery 266, 149-157 (2017).
9. M. Buitinga, F. Assen, M. Hanegraaf, P. Wieringa, J. Hilderink, L. Moroni, R. Truckenmuller, C. van Blitterswijk, G. W. Romer, F. Carlotti, E. de Koning, M. Karperien, A. van Apeldoorn, Micro-fabricated scaffolds lead to efficient remission of diabetes in mice. Biomaterials 135, 10-22 (2017).
10. J. C. Stendahl, L.-J. Wang, L. W. Chow, D. B. Kaufman, S. I. Stupp, Growth factor delivery from self-assembling nanofibers to facilitate islet transplantation. Transplantation 86, 478 (2008).
11. M. Razavi, R. Primavera, B. D. Kevadiya, J. Wang, P. Buchwald, A. S. Thakor, A Collagen Based Cryogel Bioscaffold that Generates Oxygen for Islet Transplantation. Advanced Functional Materials, 1902463 (2020).
12. S. Hu, R. Primavera, M. Razavi, A. Avadhani, J. Wang, A. S. Thakor, Hybrid Polydimethylsiloxane Bioscaffold-Intravascular Catheter for Cellular Therapies. ACS Applied Bio Materials 3, 6626-6632 (2020).
13. R. F. Gibly, X. Zhang, W. L. Lowe Jr, L. D. Shea, Porous scaffolds support extrahepatic human islet transplantation, engraftment, and function in mice. Cell transplantation 22, 811-819 (2013).
14. P. de Vos, A. F. Hamel, K. Tatarkiewicz, Considerations for successful transplantation of encapsulated pancreatic islets. Diabetologia 45, 159-173 (2002).
15. S.-M. Lee, J.-H. Kim, J.-H. Ahn, Graphene as a flexible electronic material: mechanical limitations by defect formation and efforts to overcome. Materials Today 18, 336-344 (2015).
16. M. Rosillo-Lopez, C. G. Salzmann, A simple and mild chemical oxidation route to high-purity nano-graphene oxide. Carbon 106, 56-63 (2016).
17. F. Banhart, J. Kotakoski, A. V. Krasheninnikov, Structural defects in graphene. ACS nano 5, 26-41 (2011).
18. S. Syama, P. Mohanan, Safety and biocompatibility of graphene: A new generation nanomaterial for biomedical application. International journal of biological macromolecules 86, 546-555 (2016).
19. J. M. Anderson, A. Rodriguez, D. T. Chang, in Seminars in immunology. (Elsevier, 2008), vol. 20, pp. 86-100.
20. R. Khan, R. Nakagawa, B. Campeon, Y. Nishina, A Simple and Robust Functionalization of Graphene for Advanced Energy Devices. ACS Applied Materials & Interfaces 12, 12736-12742 (2020).
21. J. P. Tuckermann, A. Kleiman, K. G. McPherson, H. M. Reichardt, Molecular mechanisms of glucocorticoids in the control of inflammation and lymphocyte apoptosis. Critical reviews in clinical laboratory sciences 42, 71-104 (2005).
22. S. Franz, S. Rammelt, D. Scharnweber, J. C. Simon, Immune responses to implants—a review of the implications for the design of immunomodulatory biomaterials. Biomaterials 32, 6692-6709 (2011).
23. W. S. Zawalich, G. J. Tesz, H. Yamazaki, K. C. Zawalich, W. Philbrick, Dexamethasone suppresses phospholipase C activation and insulin secretion from isolated rat islets. Metabolism 55, 35-42 (2006).
24. G. Ren, M. Rezaee, M. Razavi, A. Taysir, J. Wang, A. S. Thakor, Adipose tissue-derived mesenchymal stem cells rescue the function of islets transplanted in sub-therapeutic numbers via their angiogenic properties. Cell and tissue research 376, 353-364 (2019).
25. P. O. Carlsson, P. Liss, A. Andersson, L. Jansson, Measurements of oxygen tension in native and transplanted rat pancreatic islets. Diabetes 47, 1027-1032 (1998).
26. S. J. Persaud, A. C. Hauge-Evans, P. M. Jones, “Insulin-Secreting Cell Lines: Potential for Research and Diabetes Therapy” in Cellular Endocrinology in Health and Disease (Elsevier, 2014), pp. 239-256.
27. M. Razavi, F. Zheng, A. Telichko, M. Ullah, J. Dahl, A. S. Thakor, Effect of Pulsed Focused Ultrasound on the Native Pancreas. Ultrasound Med Biol 46, 630-638 (2020).
28. M. Razavi, F. Zheng, A. Telichko, J. Wang, G. Ren, J. Dahl, A. S. Thakor, improving the function and engraftment of transplanted pancreatic islets Using pulsed focused Ultrasound therapy. Scientific reports 9, 1-12 (2019).
29. G. P. Otto, B. Rathkolb, M. A. Oestereicher, C. J. Lengger, C. Moerth, K. Micklich, H. Fuchs, V. Gailus-Durner, E. Wolf, M. H. de Angelis, Clinical chemistry reference intervals for C57BL/6J, C57BL/6N, and C3HeB/FeJ mice (Mus musculus). Journal of the American Association for Laboratory Animal Science 55, 375-386 (2016).
30. O. Boehm, B. Zur, A. Koch, N. Tran, R. Freyenhagen, M. Hartmann, K. Zacharowski, Clinical chemistry reference database for Wistar rats and C57/BL6 mice. Biological chemistry 388, 547-554 (2007).
31. A. E. Jakus, E. B. Secor, A. L. Rutz, S. W. Jordan, M. C. Hersam, R. N. Shah, Three-dimensional printing of high-content graphene scaffolds for electronic and biomedical applications. ACS Nano 9, 4636-4648 (2015).
32. A. M. Davalli, L. Scaglia, D. H. Zangen, J. Hollister, S. Bonner-Weir, G. C. Weir, Vulnerability of islets in the immediate posttransplantation period. Dynamic changes in structure and function. Diabetes 45, 1161-1167 (1996).
33. A. Citro, E. Cantarelli, L. Piemonti, Anti-inflammatory strategies to enhance islet engraftment and survival. Curr Diab Rep 13, 733-744 (2013).
34. G. Reina, J. M. Gonzalez-Dominguez, A. Criado, E. Vazquez, A. Bianco, M. Prato, Promises, facts and challenges for graphene in biomedical applications. Chemical Society Reviews 46, 4400-4416 (2017).
35. S. Tedesco, C. Bolego, A. Toniolo, A. Nassi, G. P. Fadini, M. Locati, A. Cignarella, Phenotypic activation and pharmacological outcomes of spontaneously differentiated human monocyte-derived macrophages. Immunobiology 220, 545-554 (2015).
36. S. M. Abraham, T. Lawrence, A. Kleiman, P. Warden, M. Medghalchi, J. Tuckermann, J. Saklatvala, A. R. Clark, Antiinflammatory effects of dexamethasone are partly dependent on induction of dual specificity phosphatase 1. The Journal of experimental medicine 203, 1883-1889 (2006).
37. J. A. Polderman, V. Farhang-Razi, S. Van Dieren, P. Kranke, J. H. DeVries, M. W. Hollmann, B. Preckel, J. Hermanides, Adverse side effects of dexamethasone in surgical patients. Cochrane Database of Systematic Reviews, (2018).
38. N. Waldron, C. Jones, T. Gan, T. Allen, A. Habib, Impact of perioperative dexamethasone on postoperative analgesia and side-effects: systematic review and meta-analysis. British journal of anaesthesia 110, 191-200 (2013).
39. M. Razavi, S. Hu, A. S. Thakor, A collagen based cryogel bioscaffold coated with nanostructured polydopamine as a platform for mesenchymal stem cell therapy. Journal of Biomedical Materials Research Part A 106, 2213-2228 (2018).
40. M. Razavi, A. S. Thakor, An oxygen plasma treated poly(dimethylsiloxane) bioscaffold coated with polydopamine for stem cell therapy. J Mater Sci Mater Med 29, 54 (2018).
41. A. Rafacho, V. A. Giozzet, A. C. Boschero, J. R. Bosqueiro, Functional alterations in endocrine pancreas of rats with different degrees of dexamethasone-induced insulin resistance. Pancreas 36, 284-293 (2008).
42. H. Sakoda, T. Ogihara, M. Anai, M. Funaki, K. lnukai, H. Katagiri, Y. Fukushima, Y. Onishi, H. Ono, M. Fujishiro, Dexamethasone-induced insulin resistance in 3T3-L1 adipocytes is due to inhibition of glucose transport rather than insulin signal transduction. Diabetes 49, 1700-1708 (2000).
43. A. Rafacho, L. Roma, S. Taboga, A. Boschero, J. R. Bosqueiro, Dexamethasone-induced insulin resistance is associated with increased connexin 36 mRNA and protein expression in pancreatic rat islets. Canadian journal of physiology and pharmacology 85, 536-545 (2007).
44. S. Gremlich, R. Roduit, B. Thorens, Dexamethasone induces posttranslational degradation of GLUT2 and inhibition of insulin secretion in isolated pancreatic beta cells. Comparison with the effects of fatty acids. J Biol Chem 272, 3216-3222 (1997).
45. A. Ogawa, J. H. Johnson, M. Ohneda, C. T. McAllister, L. Inman, T. Alam, R. H. Unger, Roles of insulin resistance and beta-cell dysfunction in dexamethasone-induced diabetes. J Clin Invest 90, 497-504 (1992).
46. R. H. Unger, Diabetic hyperglycemia: link to impaired glucose transport in pancreatic beta cells. Science 251, 1200-1205 (1991).
47. C. Wu, R. Miron, A. Sculean, S. Kaskel, T. Doert, R. Schulze, Y. Zhang, Proliferation, differentiation and gene expression of osteoblasts in boron-containing associated with dexamethasone deliver from mesoporous bioactive glass scaffolds. Biomaterials 32, 7068-7078 (2011).
48. A. Etxabide, J. Long, P. Guerrero, K. de la Caba, A. Seyfoddin, 3D printed lactose-crosslinked gelatin scaffolds as a drug delivery system for dexamethasone. European Polymer Journal 114, 90-97 (2019).
49. A. Martins, A. R. Duarte, S. Faria, A. P. Marques, R. L. Reis, N. M. Neves, Osteogenic induction of hBMSCs by electrospun scaffolds with dexamethasone release functionality. Biomaterials 31, 5875-5885 (2010).
50. C. Hajmrle, N. Smith, A. F. Spigelman, X. Dai, L. Senior, A. Bautista, M. Ferdaoussi, P. E. MacDonald, Interleukin-1 signaling contributes to acute islet compensation. JCI insight 1, (2016).
51. C. A. Dinarello, Interleukin-18, a proinflammatory cytokine. Eur Cytokine Netw 11, 483-486 (2000).
52. K. C. Osum, A. L. Burrack, T. Martinov, N. L. Sahli, J. S. Mitchell, C. G. Tucker, K. E. Pauken, K. Papas, B. Appakalai, J. A. Spanier, B. T. Fife, Interferon-gamma drives programmed death-ligand 1 expression on islet beta cells to limit T cell function during autoimmune diabetes. Sci Rep 8, 8295 (2018).
53. H. Blomeier, X. Zhang, C. Rives, M. Brissova, E. Hughes, M. Baker, A. C. Powers, D. B. Kaufman, L. D. Shea, W. L. Lowe Jr, Polymer scaffolds as synthetic microenvironments for extrahepatic islet transplantation. Transplantation 82, 452 (2006).
54. R. F. Gibly, X. Zhang, M. L. Graham, B. J. Hering, D. B. Kaufman, W. L. Lowe Jr, L. D. Shea, Extrahepatic islet transplantation with microporous polymer scaffolds in syngeneic mouse and allogeneic porcine models. Biomaterials 32, 9677-9684 (2011).
55. K. Jiang, J. D. Weaver, Y. Li, X. Chen, J. Liang, C. L. Stabler, Local release of dexamethasone from macroporous scaffolds accelerates islet transplant engraftment by promotion of anti-inflammatory M2 macrophages. Biomaterials 114, 71-81 (2017).
56. J. Hernandez-Rodriguez, M. Segarra, C. Vilardell, M. Sanchez, A. Garcia-Martinez, M. Esteban, C. Queralt, J. Grau, A. Urbano-Marquez, A. Palacin, Tissue production of pro-inflammatory cytokines (IL-1β, TNFα and IL-6) correlates with the intensity of the systemic inflammatory response and with corticosteroid requirements in giant-cell arteritis. Rheumatology 43, 294-301 (2004).
57. H. Muhl, Pro-inflammatory signaling by IL-10 and IL-22: bad habit stirred up by interferons? Frontiers in immunology 4, 18 (2013).
58. A. El-Wakkad, M. Hassan Nel, H. Sibaii, S. R. EI-Zayat, Proinflammatory, anti-inflammatory cytokines and adiponkines in students with central obesity. Cytokine 61, 682-687 (2013).
59. K. Popko, E. Gorska, A. Stelmaszczyk-Emmel, R. Plywaczewski, A. Stoklosa, D. Gorecka, B. Pyrzak, U. Demkow, Proinflammatory cytokines 11-6 and TNF-alpha and the development of inflammation in obese subjects. Eur J Med Res 15 Suppl 2, 120-122 (2010).
60. A. M. Shapiro, C. Ricordi, B. J. Hering, H. Auchincloss, R. Lindblad, R. P. Robertson, A. Secchi, M. D. Brendel, T. Berney, D. C. Brennan, E. Cagliero, R. Alejandro, E. A. Ryan, B. DiMercurio, P. Morel, K. S. Polonsky, J. A. Reems, R. G. Bretzel, F. Bertuzzi, T. Froud, R. Kandaswamy, D. E. Sutherland, G. Eisenbarth, M. Segal, J. Preiksaitis, G. S. Korbutt, F. B. Barton, L. Viviano, V. Seyfert-Margolis, J. Bluestone, J. R. Lakey, International trial of the Edmonton protocol for islet transplantation. N Engl J Med 355, 1318-1330 (2006).
61. C. Schmidt. (Nature Publishing Group, 2017).
62. D. M. Berman, R. D. Molano, C. Fotino, U. Ulissi, J. Gimeno, A. J. Mendez, N. M. Kenyon, N. S. Kenyon, D. M. Andrews, C. Ricordi, Bioengineering the endocrine pancreas: intraomental islet transplantation within a biologic resorbable scaffold. Diabetes 65, 1350-1361 (2016).
63. R. Chang, G. Faleo, H. A. Russ, A. V. Parent, S. K. Elledge, D. A. Bernards, J. L. Allen, K. Villanueva, M. Hebrok, Q. Tang, Nanoporous immunoprotective device for stem-cell-derived β-cell replacement therapy. ACS nano 11, 7747-7757 (2017).
64. L. Davis, Basic methods in molecular biology (Elsevier, 2012).
65. J. Walker, S. Shadanbaz, N. T. Kirkland, E. Stace, T. Woodfield, M. P. Staiger, G. J. Dias, Magnesium alloys: predicting in vivo corrosion with in vitro immersion testing. Journal of Biomedical Materials Research Part B: Applied Biomaterials 100, 1134-1141 (2012).
66. F. Witte, J. Fischer, J. Nellesen, H. A. Crostack, V. Kaese, A. Pisch, F. Beckmann, H. Windhagen, In vitro and in vivo corrosion measurements of magnesium alloys. Biomaterials 27, 1013-1018 (2006).
67. J. Reifenrath, A. Marten, N. Angrisani, R. Eifler, A. Weizbauer, In vitro and in vivo corrosion of the novel magnesium alloy Mg—La—Nd—Zr: influence of the measurement technique and in vivo implant location. Biomedical Materials 10, 045021 (2015).
68. Z. Chen, W. Ren, L. Gao, B. Liu, S. Pei, H.-M. Cheng, Three-dimensional flexible and conductive interconnected graphene networks grown by chemical vapour deposition. Nature materials 10, 424 (2011).
69. L. Jia, F. Han, H. Wang, C. Zhu, Q. Guo, J. Li, Z. Zhao, Q. Zhang, X. Zhu, B. Li, Polydopamine-assisted surface modification for orthopaedic implants. Journal of orthopaedic translation 17, 82-95 (2019).
70. X. Liu, J. Cao, H. Li, J. Li, Q. Jin, K. Ren, J. Ji, Mussel-inspired polydopamine: a biocompatible and ultrastable coating for nanoparticles in vivo. ACS nano 7, 9384-9395 (2013).
71. M. Razavi, T. Ren, F. Zheng, A. Telichko, J. Wang, J. J. Dahl, U. Demirci, A. S. Thakor, Facilitating islet transplantation using a three-step approach with mesenchymal stem cells, encapsulation, and pulsed focused ultrasound. Stem Cell Research & Therapy 11, 1-17 (2020).
72. E. Song, S. Y. Kim, T. Chun, H.-J. Byun, Y. M. Lee, Collagen scaffolds derived from a marine source and their biocompatibility. Biomaterials 27, 2951-2961 (2006).
73. M. Razavi, R. Primavera, B. D. Kevadiya, J. Wang, M. Ullah, P. Buchwald, A. S. Thakor, Controlled Nutrient Delivery to Pancreatic Islets Using Polydopamine-Coated Mesoporous Silica Nanoparticles. Nano Letters 20, 7220-7229 (2020).
74. E. Cantarelli, A. Citro, S. Marzorati, R. Melzi, M. Scavini, L. Piemonti, Murine animal models for preclinical islet transplantation: no model fits all (research purposes). Islets 5, 79-86 (2013).
75. M. M. Coronel, R. Geusz, C. L. Stabler, Mitigating hypoxic stress on pancreatic islets via in situ oxygen generating biomaterial. Biomaterials 129, 139-151 (2017).
76. E. Pedraza, M. M. Coronel, C. A. Fraker, C. Ricordi, C. L. Stabler, Preventing hypoxia-induced cell death in beta cells and islets via hydrolytically activated, oxygen-generating biomaterials. Proc Natl Acad Sci USA 109, 4245-4250 (2012).
77. R. Primavera, M. Razavi, B. D. Kevadiya, J. Wang, A. Vykunta, D. Di Mascolo, P. Decuzzi, A. S. Thakor, Enhancing islet transplantation using a biocompatible collagen-PDMS bioscaffold enriched with dexamethasone-microplates. Biofabrication 13, 035011 (2021).
78. G. Wilson, E. Leiter, Streptozotocin interactions with pancreatic β cells and the induction of insulin-dependent diabetes. The Role of Viruses and the Immune System in Diabetes Mellitus, 27-54 (1990).
79. A. Molven, J. Hollister-Lock, J. Hu, R. Martinez, P. R. Njolstad, C. W. Liew, G. Weir, R. N. Kulkarni, The Hypoglycemic Phenotype Is Islet Cell—Autonomous in Short-Chain Hydroxyacyl-CoA Dehydrogenase—Deficient Mice. Diabetes 65, 1672-1678 (2016).
80. H. Yang, J. R. Wright Jr, Human β cells are exceedingly resistant to streptozotocin in vivo. Endocrinology 143, 2491-2495 (2002).

1. A bioscaffold comprising:
a) a three-dimensional graphene matrix, wherein the graphene matrix comprises a plurality of macropores and micropores; and
b) a coating comprising dexamethasone.

2. The bioscaffold of claim 1, further comprising a polydopamine nanolayer on the surface of the graphene matrix, wherein the polydopamine nanolayer is functionalized with the dexamethasone.

3-4. (canceled)

5. The bioscaffold of claim 1, wherein the macropores have an average diameter ranging from about 400 μm to about 800 μm, the micropores have an average diameter ranging from about 100 μm to about 200 μm, and the graphene matrix has a porosity ranging from 55 percent to 95 percent, as measured by scanning electron microscopy, and the bioscaffold has a thickness of from about 0.1 mm to about 25 mm.

6-7. (canceled)

8. The bioscaffold of claim 1, wherein the dexamethasone is at a concentration of about 0.25 to about 1 weight/volume percent (w/v %) in the bioscaffold.

9-10. (canceled)

11. The bioscaffold of claim 1, further comprising one or more drugs, growth factors, angiogenic agents, cytokines, or extracellular matrix components, or a combination thereof.

12. The bioscaffold of claim 1, wherein the bioscaffold further comprises therapeutic cells or extracellular vesicles, wherein the therapeutic cells or extracellular vesicles are contained in the macropores.

13. The bioscaffold of claim 12, wherein the therapeutic cells are stem cells, progenitor cells, or mature cells.

14. The bioscaffold of claim 13, wherein the stem cells are induced-pluripotent stem cells or adult stem cells.

15. The bioscaffold of claim 13, wherein the stem cells are mesenchymal stem cells.

16. (canceled)

17. The bioscaffold of claim 12, wherein the therapeutic cells secrete a cytokine, a chemokine, an antibody, an enzyme, a growth factor, or a hormone.

18. The bioscaffold of claim 17, wherein the therapeutic cells are endocrine cells, exocrine cells, stem cells, lymphocytes, or genetically modified cells.

19. (canceled)

20. The bioscaffold of claim 12, wherein the therapeutic cells are insulin-secreting cells.

21. The bioscaffold of claim 20, wherein the insulin-secreting cells are pancreatic beta cells, islets obtained from a donor, or insulin-secreting cells derived from stem cells or pancreatic progenitor cells.

22-24. (canceled)

25. The bioscaffold of claim 12, wherein the extracellular vesicles are exosomes, ectosomes, microvesicles, or microparticles derived from a plasma membrane of a cell.

26. The bioscaffold of claim 12, wherein the extracellular vesicles are derived from mesenchymal stem cells.

27. A method of treating a subject for type 1 diabetes or hyperglycemia, the method comprising implanting the bioscaffold of claim 20 in the subject at an implantation site.

28. The method of claim 27, wherein the bioscaffold releases dexamethasone for at least 2 weeks at the implantation site in vivo.

29. The method of claim 27, wherein the insulin-secreting cells are autologous, allogeneic, or xenogeneic pancreatic beta cells or islets, or the insulin-secreting cells are derived from stem cells or pancreatic progenitor cells.

30. (canceled)

31. The method of claim 27, wherein the implantation site is in a kidney, liver, omentum, peritoneum, abdomen, submuscular tissue, or subcutaneous tissue of the subject.

32-38. (canceled)

39. A method for making a graphene bioscaffold of claim 2, the method comprising:
a) fabricating a three-dimensional graphene matrix using template-directed chemical vapor deposition;
b) coating the surface of the graphene matrix with polydopamine; and
c) functionalizing the polydopamine coating with dexamethasone.

40. The method of claim 39, further comprising depositing therapeutic cells or extracellular vesicles on the bioscaffold.

41-53. (canceled)
